(19) United States
(12) Patent Application Publication
(10) Pub. No.: US 2010/0036495 A1
(43) Pub. Date: Feb. 11, 2010

(54) Device and method for treating spine

(75) Inventors: Wolfgang Daum, Groton, MA (US); Amy Fredrick, Boston, MA (US); Mitchell Hardenbrook, Hopkinton, MA (US); Joyce Lauer, Wayland, MA (US); Kevin P. Staid, Lowell, MA (US)

Correspondence Address: JOYCE E. LAUER, 75 MOORE ROAD, WAYLAND, MA, 01778, US

(73) Assignee: PX Spine Corporation

(21) Appl. No.: 12/460,413

(22) Filed: Jul. 17, 2009

Related U.S. Application Data
(60) Provisional application No. 61/188,180, filed on Aug. 7, 2008.

(51) Int. Cl. A61F 2/44

(52) U.S. Cl. 623/17.11

(57) ABSTRACT

A device for treating a spine includes a proximal component positioned partially or entirely within a first vertebra and a distal component positioned partially or entirely within a second vertebra and may also include an intermediate component. A method for treating a spine includes forming a curved channel that extends through a first vertebra from a pedicle to an endplate, and advancing the components through the curved channel, the orientation of the proximal component relative to the distal component changing by at least 40 degrees while the proximal component passes through the curved channel. In another aspect, a method for treating a spine includes forming a curved channel that has a pedicle region, a central region, and an endplate region, where the channel diameter for the central region is larger than the channel diameter for the pedicle region or the endplate region, and advancing an implant through the channel.

### CROSS-REFERENCE TO RELATED APPLICATIONS

This application claims the benefit of U.S. Provisional Application No. 61/188,180, filed on Aug. 7, 2008. All of the above-referenced applications are incorporated by reference herein.

### BACKGROUND

The spine consists of a number of vertebrae, spinal discs between the vertebrae that act as shock absorbers, and ligaments that link the vertebrae. The vertebrae, spinal discs, and ligaments, together with associated muscles, form a strong yet flexible column. Deterioration of vertebrae or spinal discs, or altered positioning of vertebrae, may result from various conditions, injuries, or disease states. Treatment of such deterioration or altered positioning may employ devices or methods that stabilize the position of a vertebra relative to one or more other vertebrae. Stabilization may employ surgical implantation of devices or prostheses. Stabilization may also include inducing new bone to grow between vertebrae, resulting in fusion of vertebrae.

### SUMMARY

A device for treating a spine includes a proximal component positioned partially or entirely within a first vertebra and a distal component positioned partially or entirely within a second vertebra and may also include an intermediate component. A method for treating a spine includes forming a curved channel that extends through a first vertebra from a pedicle to an endplate, and advancing the components through the curved channel, the orientation of the proximal component relative to the distal component changing by at least 40 degrees while the proximal component passes through the curved channel. In another aspect, a method for treating a spine includes forming a curved channel that has a pedicle region, a central region, and an endplate region, where the channel diameter for the central region is larger than the channel diameter for the pedicle region or the endplate region, and advancing an implant through the channel.

Additional embodiments are described in the detailed description below. This summary does not purport to define the invention. The invention is defined by the claims.

### BRIEF DESCRIPTION OF THE DRAWINGS

FIG. 1 is a side view of four vertebrae in the lumbar and sacral regions of a human spine.

FIG. 2 is an axial cephalad view of vertebra L5 in the lumbar region of a human spine.

FIG. 3 is a partial section side view of two vertebrae and a device for treating a spine, the device comprising a proximal component that is anchored in a first vertebra and a distal component that is anchored in a second vertebra.

FIG. 4 is a section view of the device of FIG. 3, with the plane of section taken along line A-A′ of FIG. 3.

FIG. 5 is a section view of the individual components of the device of FIG. 3, with the plane of section for each component taken along line A-A′ of FIG. 3.

FIG. 6 is a partial section side view of two vertebrae during installation of a device for treating a spine, the device comprising a proximal component that is to be anchored in a first vertebra and a distal component that is anchored in a second vertebra, the view being taken while the proximal component passes through the curved channel.

FIG. 7 is a partial section side view of two vertebrae and a distal component passing through a curved channel.

FIG. 7B is a partial section side view of two vertebrae during installation of a device for treating a spine, the view being taken while a distal component for the device passes through the curved channel.

FIG. 7C is a section view of a proximal component that includes a proximal component recess and that has a flared surface surrounding a portion of the proximal component passage or the proximal component recess.

FIG. 7D is a section view of the proximal component of FIG. 7C inserted over a guidewire, with a driver for advancing the proximal component.

FIG. 7E is a section view of a distal component that has a flared surface surrounding a portion of the distal component passage, with a driver for advancing the distal component.

FIG. 7F is a section view of a distal component that has a flared surface surrounding a portion of the distal component passage.

FIG. 8 is a partial section side view of two vertebrae in the lumbar region of a human spine, indicating the vertebral body height, the pedicle height, the channel diameter for the pedicle region of the curved channel, and the radius of curvature for the curved region of the curved channel.

FIG. 9 is an axial cephalad view of vertebra L5 in the lumbar region of a human spine, indicating the pedicle width and the channel diameter for the pedicle region of the curved channel.

FIG. 10 is a partial section side view of two vertebrae and a device for treating a spine, the device comprising a proximal component that is anchored in a first vertebra and a distal component that is anchored in a second vertebra.

FIG. 11 is a section view of the device of FIG. 10, with the plane of section taken along line A-A′ of FIG. 10.

FIG. 12 is a section view of the individual components of the device of FIG. 10, with the plane of section for each component taken along line A-A′ of FIG. 10.

FIG. 13 is a partial section side view of two vertebrae and a device for treating a spine, the device comprising a proximal component that is anchored in a first vertebra and a distal component that is anchored in a second vertebra.

FIG. 14 is a partial section anterior view of the bodies of two vertebrae and plural devices for treating a spine, each device comprising a proximal component that is anchored in a first vertebra and a distal component that is anchored in a second vertebra.

FIG. 15 is a partial section side view of two vertebrae and tools used in a method for treating a spine, during the forming of a curved channel in a first vertebra.

FIG. 16 is a partial section side view of two vertebrae and tools used in a method for treating a spine, during the forming of a curved channel in a first vertebra.

FIG. 17 is a partial section side view of two vertebrae and tools used in a method for treating a spine, during the forming of a curved channel in a first vertebra.

FIG. 18 is a partial section side view of two vertebrae and tools and a distal component used in a method for treating a spine, during the advancing of the distal component through the curved channel.

FIG. 19 is a partial section side view of two vertebrae and tools and a distal component and a proximal component used in a method for treating a spine, during the advancing of the proximal component through the curved channel.

FIG. 20 depicts a steerable needle that may be used in forming a curved channel.

FIG. 21 depicts a steerable drilling tool that may be used in forming a curved channel.

FIG. 22A is a partial section side view of a first vertebra and a second vertebra during the performance of a method that includes forming a channel, the channel having a channel diameter, a pedicle region, a central region, and an endplate region, wherein the channel diameter for the central region is greater than the channel diameter for the pedicle region and the channel diameter for the central region is greater than the channel diameter for the endplate region.

FIG. 22B depicts the retractable cutting head FIG. 22A.

FIG. 23 is a partial section side view of a first vertebra and a second vertebra during the performance of a method that includes forming a channel, the channel having a channel diameter, a pedicle region, a central region, and an endplate region, wherein the channel diameter for the central region is greater than the channel diameter for the pedicle region and the channel diameter for the central region is greater than the channel diameter for the endplate region.

FIG. 24 is a partial section side view of a first vertebra and a second vertebra during the performance of a method that includes forming a channel, the channel having a channel diameter, a pedicle region, a central region, and an endplate region, wherein the channel diameter for the central region is greater than the channel diameter for the pedicle region and the channel diameter for the central region is greater than the channel diameter for the endplate region.

### DETAILED DESCRIPTION

Reference will now be made in detail to some embodiments, examples of which are illustrated in the accompanying drawings. In this description and in the appended claims, the terms ‘a’ or ‘an’ are used, as is common in patent documents, to include one or more than one. In this description and in the appended claims, the term ‘or’ is used to refer to a nonexclusive or, unless otherwise indicated.

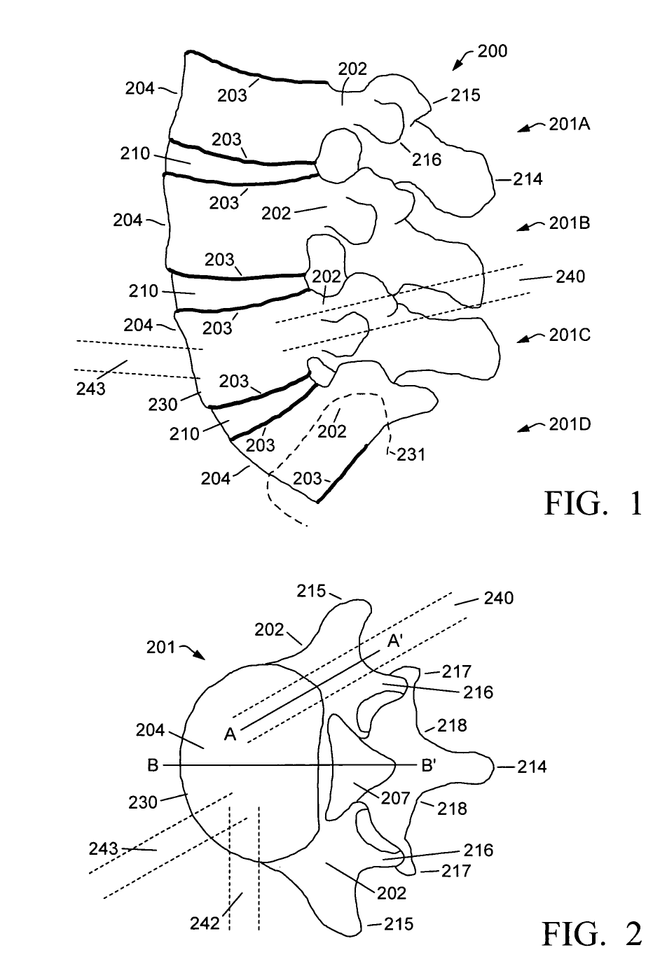

FIG. 1 is a side view of four vertebrae 201A, 201B, 201C and 201D in the lumbar and sacral regions of a human spine 200. The depicted vertebrae 201A, 201B, 201C and 201D correspond to human vertebrae L3, L4, L5, and S1, respectively. FIG. 2 is an axial cephalad view of vertebra L5. Each vertebra 201 includes an anterior part, the body 204, and a posterior part, the vertebral arch, that consists of a pair of pedicles 202 and a pair of laminae 218. The body 204, the pedicles 202, and the laminae 218 together enclose an opening, the vertebral foramen 207; the spinal cord passes through the vertebral foramen 207. The vertebral arch supports seven processes: a spinous process 214, a pair of transverse processes 215, a pair of superior articular processes 216, and a pair of inferior articular processes 217. The first sacral (S1) vertebra 201D includes a portion of the auricular surface 231 of the sacrum.

The body 204 is composed of cancellous bone covered by a thin layer of cortical bone. Cortical bone is strong and compact, while cancellous bone is more cellular and has many apertures, so that it is less strong than cortical bone. Spinal discs (intervertebral discs) 210 located between the vertebral bodies 204 serve as shock absorbers that cushion the bodies 204. Each body 204 has two endplates 203, one on the superior (upper or cephalad) surface of the body 204, and one on the inferior (lower or caudal) surface of the body 204. A body wall 230 made of cortical bone extends between the superior and inferior endplates 203. The endplates 203 are made of cortical bone. The endplate 203 has a thickness of about one to several millimeters. External to the endplate 203 is a layer of cartilage. Blood vessels in the cartilage supply nutrients to the adjacent spinal disc 210.

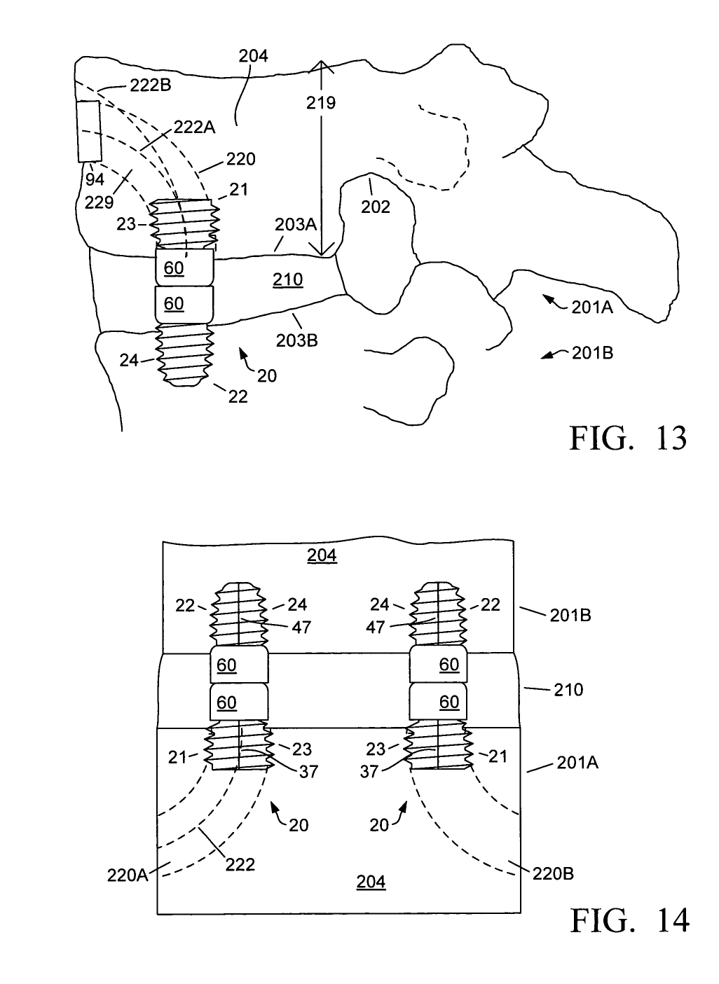

Surgical procedures for the spine 202 may employ various surgical approaches such as an anterior approach 243 or a posterior approach 240 or a lateral approach 242. These various surgical approaches are indicated by paired dashed lines in FIGS. 1 and 2. A transpedicular posterior approach 240 through a pedicle 202 is indicated in FIGS. 1 and 2. An embodiment that employs an anterior approach 243 is depicted in FIG. 13, and an embodiment that employs a lateral approach 242 is depicted in FIG. 14. Embodiments employing a transpedicular posterior approach 240 are depicted in FIGS. 3, 6, 7, 8, 10, and 15-19.

FIG. 3 is a partial section side view of two vertebrae 201A, 201B and a device 20 for treating a spine, the device 20 comprising a proximal component 21 that is anchored in a first vertebra 201A and a distal component 22 that is anchored in a second vertebra 201B, in accordance with an embodiment. In FIG. 3 and several other Figures herein, the device 20 is depicted in side view and the vertebrae 201 are depicted in partial section view. For clarity in FIG. 3 and other Figures herein, vertebrae 201 are shown as silhouettes with minimal detail of surface features or internal features of the vertebrae 201. The embodiment of FIG. 3 further comprises an intermediate component 60. Also depicted in FIG. 3 is a silhouette labelled 21A which indicates the location and orientation of proximal component 21 at an earlier time during installation of device 20; the orientation of proximal component 21 is discussed further in connection with FIG. 6.

The first vertebra 201A has a first endplate 203A that is adjacent a spinal disc 210. The second vertebra 201B has a second endplate 203B that is adjacent the spinal disc 210. As used herein and in the appended claims, the term “spinal disc 210” means a normal spinal disc that is not injured or diseased and that has not been manipulated surgically and also means a spinal disc that has been injured or diseased or manipulated surgically so that some or all of the tissue between the first endplate 203A and the second endplate 203B has been removed or altered. The first vertebra 201A has a first pedicle 202 and a body wall 230.

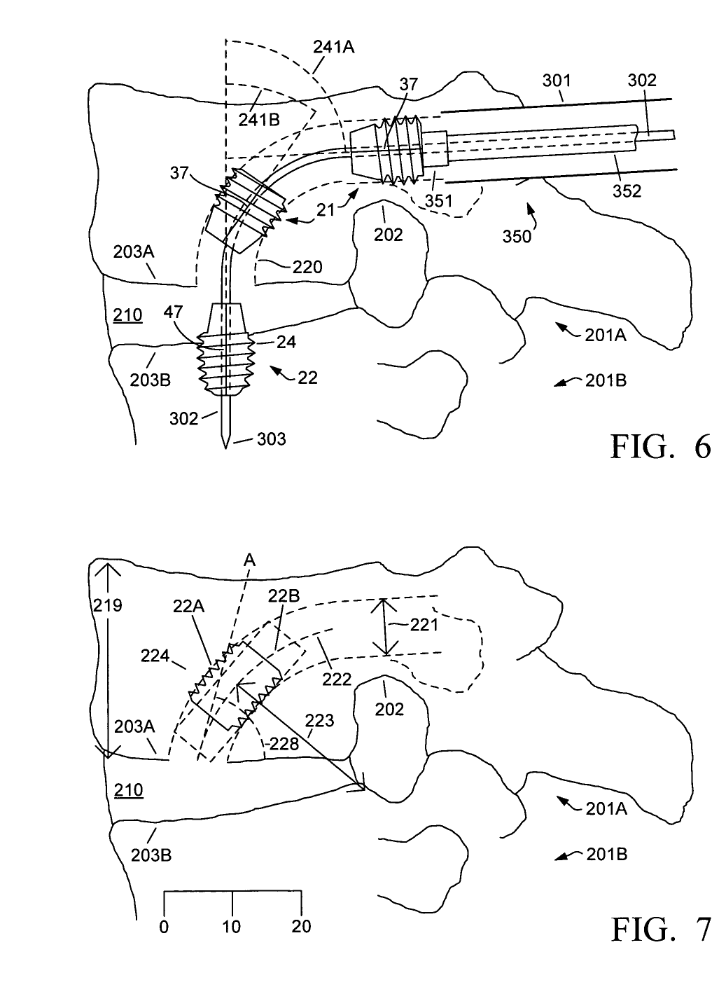

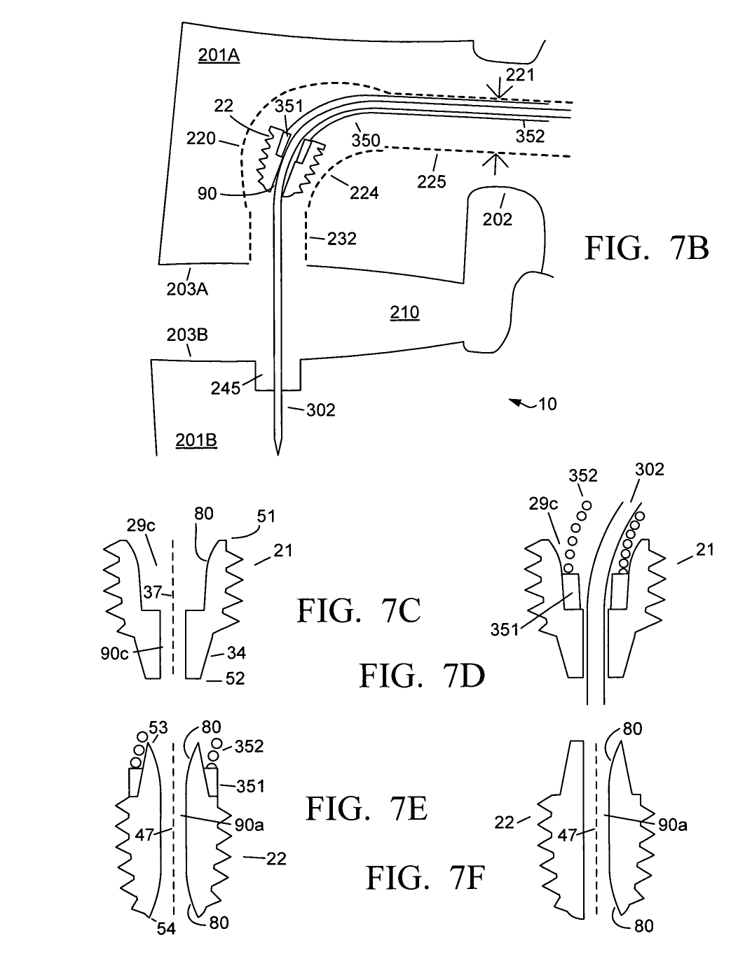

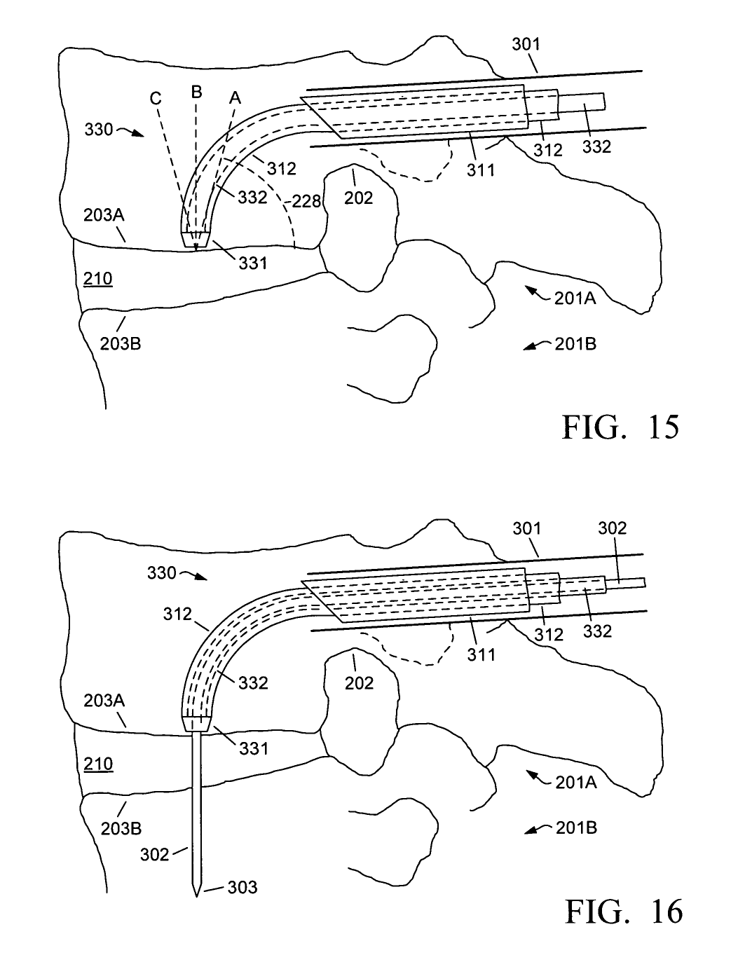

A curved channel 220 extends through the first vertebra 201A from the first pedicle 202 or the body wall 230 to the first endplate 203A. In the embodiment of FIG. 3, which employs a posterior approach 240, the curved channel 220 includes a pedicle region 225 and a curved region 224. In embodiments that employ an anterior approach 243 or a lateral approach 242, such as the embodiments of FIGS. 13 or 14, the curved channel 220 includes a curved region 224 but does not include a pedicle region 225. The channel diameter 221 is selected in relation to the dimensions for an individual vertebra 201, as described in connection with FIGS. 7, 8, and 9. The curved channel 220 may be formed as described in connection with FIGS. 15-19.

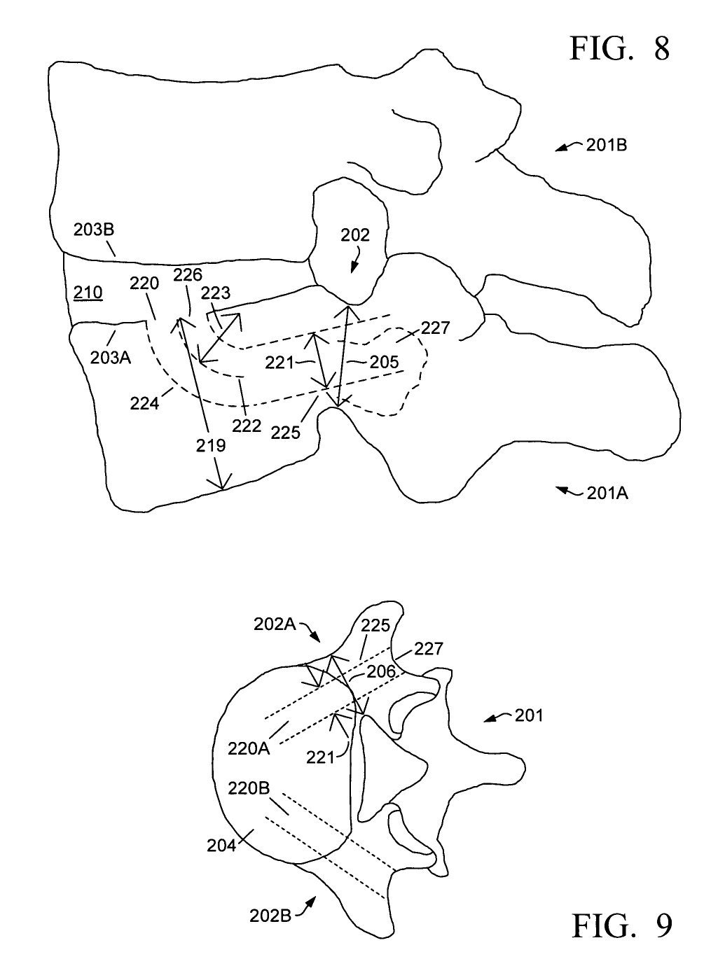

As used herein and in the appended claims, the term “curved channel” means that a curved region 224 for curved channel 220 has a radius of curvature 223 that is less than or equal to 100 percent of a vertebral body height 219 for first vertebra 201A. Radius of curvature 223 for the curved region 224 is described in more detail in connection with FIG. 8.

Device 20, anchored in first vertebra 201A and in second vertebra 201B, provides a sturdy support that may be used to stabilize the vertebrae 201. Device 20 may be used independently for stabilization, or it may be used for stabilization in a procedure for fusion of vertebrae 201A and 201B using bone graft material or other fusion substrates. In some embodiments, device 20 may also be used to distract vertebrae 201A and 201B.

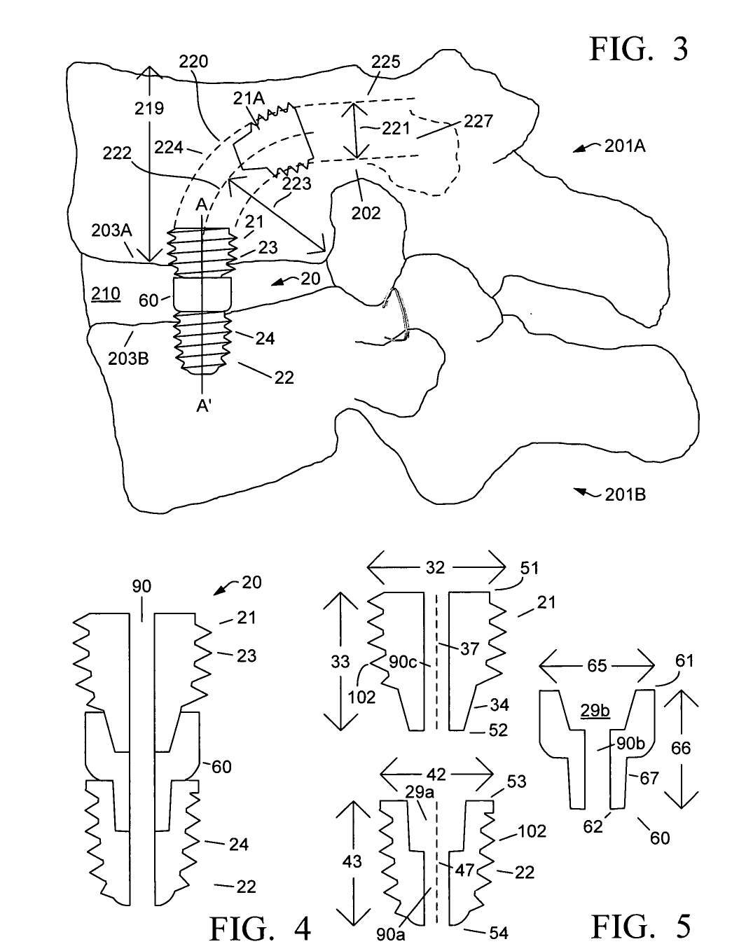

FIG. 4 is a section view of the device 20 of FIG. 3, with the plane of section taken along line A-A′ of FIG. 3. FIG. 5 is a section view of the individual components of the device 20 of FIG. 3, with the plane of section for each component taken along line A-A′ of FIG. 3.

The device 20 of the FIG. 3-5 embodiment comprises a proximal component 21, an intermediate component 60, and a distal component 22. Proximal component 21 has a proximal component first end 51 and a proximal component second end 52. Proximal component 21 comprises a proximal component tapered region 34 adjacent the proximal component second end 52 and a first thread 102 for anchoring in the first vertebra 201A. Proximal component 21 defines a proximal component passage 90c that is capable of receiving a guidewire 302, the proximal component passage 90c extending from the proximal component first end 51 to the proximal component second end 52. Guidewire 302 is depicted in FIG. 6 and other Figures herein.

Intermediate component 60 has an intermediate component first end 61 and an intermediate component second end 62. Intermediate component 60 comprises an intermediate component tapered region 67 adjacent the intermediate component second end 62. Intermediate component 60 defines an intermediate component passage 90b that is capable of receiving the guidewire 302, the intermediate component passage 90b extending from the intermediate component first end 61 to the intermediate component second end 62. Intermediate component 60 defines an intermediate component recess 29b adjacent the intermediate component first end 61, the intermediate component recess 29b being coaxial with the intermediate component passage 90b.

Distal component 22 has a distal component first end 53 and a distal component second end 54. Distal component 22 comprises a second thread 102 for anchoring in the second vertebra 201B. Distal component 22 defines a distal component passage 90a that is capable of receiving the guidewire 302, the distal component passage 90a extending from the distal component first end 53 to the distal component second end 54. Distal component 22 defines a distal component recess 29a adjacent the distal component first end 53, the distal component recess 29a being coaxial with the distal component passage 90a.

In the embodiment of FIGS. 3-5, the intermediate component tapered region 67 is capable of being seated within the distal component recess 29a, and the proximal component tapered region 34 is capable of being seated within the intermediate component recess 29b. Thus, intermediate component 60 releasably engages distal component 22, and proximal component 21 releasably engages intermediate component 60.

In the embodiment of FIGS. 3-5, each individual component is a single piece. In other embodiments, a component may comprise plural pieces. For example, a component may comprise an inner portion and an outer portion. As used herein and in the appended claims, the term “portion” includes separate pieces of a component and also includes separate regions within a component that is a single piece. As used herein and in the appended claims, the term “least a . . . portion” for a component means a portion of a component or the entire component.

Proximal component 21 has a proximal component position. As used herein and in the appended claims, the term “position” means the final position of a component after installation of device 20 is complete. The proximal component position is entirely within the first vertebra 201A or partially within the first vertebra 201A and partially within the spinal disc 210. Distal component 22 has a distal component position. The distal component position is entirely within the second vertebra 201B or partially within the second vertebra 201B and partially within the spinal disc 210. In the embodiment of FIGS. 3-5, proximal component 21 and distal component 22 each extend somewhat into spinal disc 210; in other words, each component is positioned partially within a vertebra 201 and partially within spinal disc 210. In another embodiment, proximal component 21 or distal component 22 may be positioned entirely instead of partially within vertebra 201A or vertebra 201B, respectively, with no extension into spinal disc 210. In such an embodiment, an intermediate component 60 may be positioned partially within spinal disc 210 and partially within a vertebra 201.

Proximal component 21 includes first means for anchoring 23 in the first vertebra 201A. Distal component 22 includes second means for anchoring 24 in the second vertebra 201B. In the embodiment of FIGS. 3-5, first means for anchoring 23 is a thread 102 and second means for anchoring 24 is a thread 102, as indicated in FIG. 5. In the embodiment of FIGS. 3-5, first means for anchoring 23 covers a significant fraction of the lateral surface of proximal component 21, so that first means for anchoring 23 engages first endplate 203A and also engages the cancellous bone that occupies the interior of body 204 of first vertebra 201A. In other words, first means for anchoring 23 includes means for engaging first endplate 203A and also includes means for engaging the cancellous bone adjacent first endplate 203A. Similarly, second means for anchoring 24 covers a significant fraction of the lateral surface of distal component 22, so that second means for anchoring 24 engages second endplate 203B and also engages the cancellous bone that occupies the interior of body 204 of second vertebra 201B. In other words, second means for anchoring 24 includes means for engaging second endplate 203B and also includes means for engaging the cancellous bone adjacent second endplate 203B.

In another embodiment, first means for anchoring 23 may include means for engaging cancellous bone in vertebra 201A but not means for engaging first endplate 203A. For example, in the embodiment of FIG. 13, proximal component 21 is positioned relatively far from spinal disc 210, so that first means for anchoring 23 has little or no engagement with first endplate 203A. In another example, thread 102 could be present on a smaller fraction of the surface of proximal component 21, compared to the embodiment of FIG. 3, the smaller fraction being located near first end 51. Similarly, second means for anchoring 24 may include means for engaging cancellous bone in vertebra 201B but not means for engaging second endplate 203B. For example, in the embodiment of FIG. 14, each of the distal components 22 is positioned relatively far from spinal disc 210, so that second means for anchoring 24 has little or no engagement with second endplate 203B.

Embodiments such as that of FIGS. 3-5 may be used to stabilize vertebrae 201 and may optionally be used to distract vertebrae 201. Installation of device 20 within vertebrae 201 is described in connection with FIGS. 15-19. Installation of device 20 includes anchoring of device 20 in vertebrae 201A, 201B using first means for anchoring 23 and second means for anchoring 24, and releasable engagement of the components with one another, so that each component is positioned correctly relative to other components, as depicted, for example, in FIG. 3. In the embodiment of FIGS. 3-5, first means for anchoring 23 is a thread 102. Proximal component 21 is rotated during installation so that thread 102 engages first vertebra 201A and so that proximal component 21 extends far enough beyond first endplate 203A to engage intermediate component 60, with proximal component 21 pushing firmly against intermediate component 60. For distraction of vertebrae 201A, 201B, proximal component 21 may be rotated further so that proximal component 21 extends further beyond first endplate 203A, thereby increasing the force exerted upon intermediate component 60, which in turn exerts force on distal component 22. The exerted force causes first endplate 203A and second endplate 203B to move apart from one another, so that the distance between the endplates 203 is increased and the vertebrae 201 are distracted.

As indicated in FIG. 5, proximal component 21 has a proximal component axis 37 that extends from proximal component first end 51 to proximal component second end 52. Distal component 22 has a distal component axis 47 that extends from distal component first end 53 to distal component second end 54. For any component, the first end for the component is the component end that is positioned nearest to the channel proximal end 227 when installation of device 20 is complete. Channel proximal end 227 and channel disc end 226 are indicated in FIGS. 3, 8, and 9. For an embodiment in which a component includes plural portions, the component axis pertains to each portion; in other words, the axis for a portion is aligned with the component axis.

Proximal component axis 37 is substantially straight, and distal component axis 47 is substantially straight. As used herein and in the appended claims, a statement that a component axis such as proximal component axis 37 or distal component axis 47 is “substantially straight” means that: (1) the component axis has a component radius of curvature that is greater than or equal to 10 centimeters; and (2) the component axis is substantially straight while the component is passing through curved channel 220 and also after completion of installation of device 20 when the component has attained its respective proximal component position or distal component position. In some embodiments, the component radius of curvature for either the proximal component axis 37 or the distal component axis 47 may be larger, resulting in a component axis that is highly straight. For example, the component radius of curvature may be greater than or equal to 12, 15, 20, 25, 30, 40, 50, or 100 centimeters.

Proximal component axis 37 is substantially perpendicular to the first endplate 203A when installation of device 20 is complete and proximal component 21 attains its proximal component position relative to first vertebra 201A and spinal disc 210. As used herein and in the appended claims, the term “substantially perpendicular” means an angle 228 having a value between 75 degrees and 105 degrees. In other words, angle 228 has a value that is greater than or equal to 75 degrees and less than or equal to 105 degrees. FIG. 15 includes three dashed lines labelled A, B, and C that intersect first endplate 203A at three different angles 228: line A intersects at 75 degrees, line B intersects at 90 degrees, and line C intersects at 105 degrees. For the proximal component 21 in the embodiment of FIG. 3, the angle 228 is about 90 degrees. In other embodiments, angle 228 may have any value between 75 degrees and 105 degrees, such as a number of degrees that is an integral or non-integral number of degrees that is greater than or equal to 75 degrees and less than or equal to 105 degrees. FIG. 7 includes a dashed line labelled A that intersects first endplate 203A at an angle 228 of about 75 degrees.

A substantially perpendicular positioning for proximal component 21 relative to first endplate 203A results in a longitudinal axis for device 20 (i.e., section line A-A′ in FIG. 3) being approximately parallel to the longitudinal axis of the spine, so that the axial physiological load on the spine is approximately parallel to the longitudinal axis of device 20.

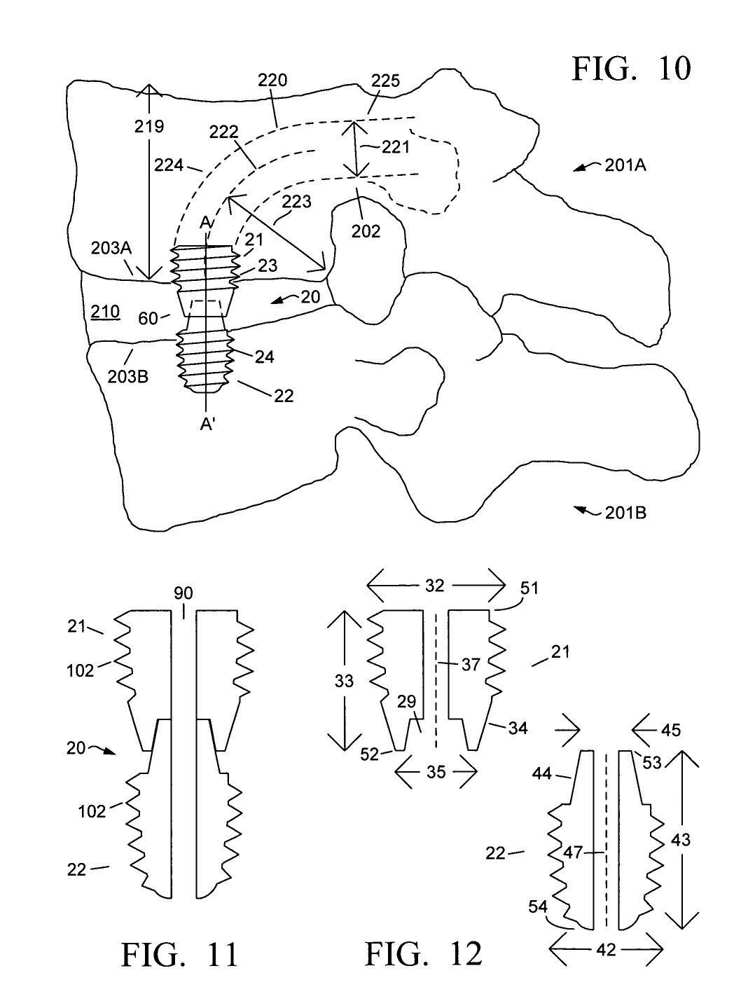

FIG. 6 is a partial section side view of two vertebrae 201A, 201B during installation of a device 20 for treating a spine, the device 20 comprising a proximal component 21 that is to be anchored in a first vertebra 201A and a distal component 22 that is anchored in a second vertebra 201B, the view being taken while the proximal component 21 passes through the curved channel 220, in accordance with an embodiment. As described in connection with FIGS. 15-19, device 20 may be installed within vertebrae 201 by passing the components or portions of components through curved channel 220. The proximal component axis 37 has an orientation relative to the distal component axis 47. During installation of the device 20 the orientation changes by at least 40 degrees while the proximal component 21 passes through the curved channel 220. As used herein and in the appended claims, the term “orientation” means an angle 241 between a proximal component axis 37 and a distal component axis 47. FIG. 6 indicates the orientation of a proximal component 21 relative to a distal component 22 at two different times while the proximal component 21 passes through the curved channel 220. The proximal component 21 and distal component 22 in the embodiment of FIG. 6 are similar to the proximal component 21 and distal component 22 in the embodiment of FIGS. 10-12.

In FIG. 6, proximal component 21 is depicted at a first location at a first time and is also depicted at a second location at a second time. At the first time, the orientation is angle 241A which equals about 87 degrees. At the second time, after the proximal component 21 has advanced further in the curved channel 220, the orientation is angle 241B which equals about 35 degrees. A difference between angle 241A and angle 241B equals 87 degrees minus 35 degrees which equals 52 degrees. In other words, the orientation (angle 241) changes by at least 20 degrees while the proximal component 21 passes through the curved channel 220. At a third time (not depicted), proximal component 21 may advance further to a location that is adjacent distal component 22 where the angle 241 is very small (less than 5 degrees), so that the total change in orientation is at least 80 degrees (87 degrees minus 5 degrees is greater than or equal to 80 degrees). In various embodiments, the orientation may change by at least 20, 25, 30, 35, 40, 45, 50, 55, 60, 65, 70, 75, 80, 85, or 90 degrees. As used herein and in the appended claims, installation means a process or method, such as that described in connection with FIGS. 15-19, that includes advancing components through the curved channel 220, where the advancing begins at the proximal end 227 of the curved channel 220 and continues in a direction towards the disc end 226.

As used herein and in the appended claims, the phrase “pass through the channel” means that a component or a portion of a component moves within curved channel 220 and eventually arrives at a position for the component or portion that is correct relative to other components or portions for the device 20. For some components, such as the distal component 22 in the embodiment of FIG. 3, the component will traverse all of curved channel 220 and then advance further to reach the second vertebra 201B. For other components or portions of a component, the component or portion may be located partially or entirely within curved channel 220 when correctly positioned relative to other components or other portions. For example, the proximal component position for proximal component 21 in the embodiment of FIG. 3 is partially within curved channel 220 within vertebra 201A and partially within the spinal disc 210.

As indicated in FIG. 5, proximal component 21 has a proximal component diameter 32 and a proximal component length 33. Distal component 22 has a distal component diameter 42 and a distal component length 43. Intermediate component 60 has an intermediate component diameter 65 and an intermediate component length 66. As used herein and in the appended claims, the term “diameter” means the transverse dimension of a region of a curved channel 220 or the transverse dimension of a component or a portion of a component; the term “diameter” is not restricted to entities having a circular cross section. The diameter for a component or portion is measured where the transverse dimension is largest. The proximal component length 33 is measured along proximal component axis 37, and the distal component length 43 is measured along distal component axis 47.

The diameter, length, and other dimensions and aspects of each component, or a portion of the component, may depend in part upon: (1) the size of individual vertebrae 201; (2) the number of devices 20 that are installed; and (3) the channel diameter 221 and the radius of curvature 223 for a curved channel 220. Thus, it may be appropriate to use larger components for vertebrae 201 that are large, and smaller components for vertebrae 201 that are small. Mean dimensions and dimension ranges for human vertebrae 201 are indicated in Table 1, which is discussed in connection with FIG. 8. As described in connection with FIG. 9 and FIG. 14, a plurality of devices 20 may be installed to stabilize a pair of vertebrae 201A and 201B, so that the plural devices 20 share the physiological load upon the vertebrae 201A and 201B. When plural devices 20 share the physiological load, it may be appropriate to use smaller component dimensions, compared to an embodiment with a single device 20 that stabilizes a pair of vertebrae 201A, 201B.

During installation of a device 20, each component passes through the curved channel 220, as described in connection with FIGS. 15-19. The channel diameter 221 and the radius of curvature 223 are relevant to the dimensions of a component that passes through the curved channel 220. FIG. 7 is a partial section side view of two vertebrae 201A, 201B and a distal component 22A passing through a curved channel 220 that includes a curved region 224, in accordance with an embodiment. For the embodiment of FIG. 7, the distal component diameter 42 is approximately equal to the channel diameter 221. In other embodiments, a smaller component diameter, relative to the channel diameter 221, may facilitate the passage of a component through curved channel 220. The distal component length 43 is restricted by the radius of curvature 223. If the distal component diameter 42 is smaller, as in the silhouette labelled 22B, then the distal component length 43 can be larger than that of distal component 22A, and still fit within the curved region 224. In other words, the diameter and length of a component interact to influence whether the component is able to pass through the curved channel 220.

If a first means for anchoring 23 or a second means for anchoring 24 includes a thread 102, rotation of the component may assist the component to pass through the curved channel 220.

The proximal component length 33 is less than or equal to 80 percent of a curved region length for the curved channel 220. The distal component length 43 is less than or equal to 80 percent of a curved region length for the curved channel 220. The curved region length is measured along the longitudinal axis 222 for channel 220 within the curved region 224. The segment of longitudinal axis 222 that is within curved region 224 is indicated in FIG. 7. For many embodiments, the proximal component length 33 or the distal component length 43 may be less than or equal to a smaller percent of the curved region length, such as 75, 70, 65, 60, 55, or 40 percent of the curved region length. For example, in the embodiment of FIG. 6, the proximal component length 33 is less than or equal to about 40 percent of the curved region length, and the distal component length 43 is less than or equal to about 50 percent of the curved region length. In the embodiment of FIG. 7, the distal component length 43 for distal component 22A is less than or equal to about 40 percent of the curved region length, and the distal component length 43 for the silhouetted alternative distal component 22B is less than or equal to about 70 percent of the curved region length.

The distal component 22A depicted in the embodiment of FIG. 7 has the same distal component diameter 42 from first end 53 to second end 54, except for small variations caused by the thread 102. In other words, distal component 22A is not narrowed or tapered. In other embodiments, a component may be narrowed or tapered, so that the diameter for the component is smaller in some portions of the component than in other portions. Narrowing or tapering of a component may facilitate passage of the component through the curved channel 220, and tapering may facilitate engagement between components during installation of device 20. Narrowing or tapering is described in connection with FIGS. 10-12.

As indicated in Table 1, the mean value for vertebral body height 219 for human lumbar vertebrae L3, L4, and L5 is 28-30 millimeters. Table 1 is described in connection with FIG. 8. A scale at the lower edge of FIG. 7 indicates lengths of 10 and 20 millimeters, for a hypothetical vertebra 201 having a vertebral body height 219 of 29 millimeters. For a vertebra 201 having a vertebral body height of 29 millimeters, and for the embodiment depicted in FIG. 7, the depicted channel diameter 221 corresponds to about 8-9 millimeters. For the depicted embodiment, the distal component diameter 42 for distal component 22A is about 8 millimeters, and the distal component length 43 for distal component 22A is about 12 millimeters. In other embodiments, the diameter and length of components may be larger or smaller than the dimensions depicted in FIG. 7. The diameter of a component typically may range from about 6 millimeters to about 17 millimeters. The length of a component typically may range from about 8 millimeters to about 25 millimeters, depending upon the size of the vertebrae 201, the radius of curvature 223, and the degree of narrowing or tapering of the component. For an embodiment that includes an intermediate component 60, as in the embodiment of FIG. 3, or plural intermediate components 60, as in the embodiments of FIGS. 13 and 14, the lengths of individual components may be smaller than for a device 20 embodiment such as that of FIGS. 10-12, which does not include any intermediate component 60.

A larger diameter or a larger length for a component increases the surface area that can support a first means for anchoring 23 or a second means for anchoring 24. Thus, larger component dimensions may improve the anchoring of proximal component 21 or distal component 22.

FIG. 7B is a partial section side view of two vertebrae 201A, 201B during installation of a device 20 for treating a spine, the view being taken while a distal component 22 for the device 20 passes through the curved channel 220. In the FIG. 7B embodiment, a channel extension 245 has been formed in second vertebra 201B, to facilitate the installation of distal component 22 at a position that is partially or entirely within second vertebra 201B. The distal component 22 is similar to that of the FIG. 3-5 embodiment and includes a distal component recess 29 a as depicted in FIG. 5. Distal component 22 is advanced through channel 220 by a driver 350 that includes a driver shaft 352 and a driver bit 351 that is inserted into distal component recess 29a.

In the FIG. 7B embodiment, the curved channel 220 includes a pedicle region 225, a curved region or central region 224, and an endplate region 232. In the embodiment of FIG. 7B, the curved channel 220 has a variable channel diameter 221. The channel diameter 221 for the central region 224 is greater than the channel diameter 221 for the pedicle region 225 of the channel 220 and greater than the channel diameter 221 for the endplate region 232 of the channel 220. As used herein and in the appended claims, the terms “curved region 224 ” and “central region 224 ” have the same meaning and are used interchangeably, and the terms “curved channel 220 ” and “channel 220 ” have the same meaning and are used interchangeably.

The variation in channel diameter 221 in the FIG. 7B embodiment serves to accommodate the different constraints upon channel diameter 221 for the pedicle region 225, the central region 224, and the endplate region 232. As described in connection with FIG. 7, the curvature of the central region 224 may impede the passage of a component that has a relatively large diameter or length. When the channel diameter 221 is increased in the central region 224, the component may be able to pass more easily through the central region 224. Furthermore, a large channel diameter 221 in the central region 224 may facilitate aligning a component so that the component can be installed substantially perpendicular to the first endplate 203A or the second endplate 203B.

In the pedicle region 225, however, a smaller channel diameter 221 may be advantageous in order to maintain the strength of the pedicle 202. A smaller channel diameter 221 may be advantageous in the endplate region 232 as well, because a smaller channel diameter 221 preserves more of the first endplate 203A and thus helps to maintain the strength of the vertebral body 204. The foregoing considerations lead to the channel 220 embodiment depicted in FIG. 7B: a channel 220 with a larger channel diameter 221 for the central region 224 and a smaller channel diameter 221 for the pedicle region 225 or the endplate region 232.

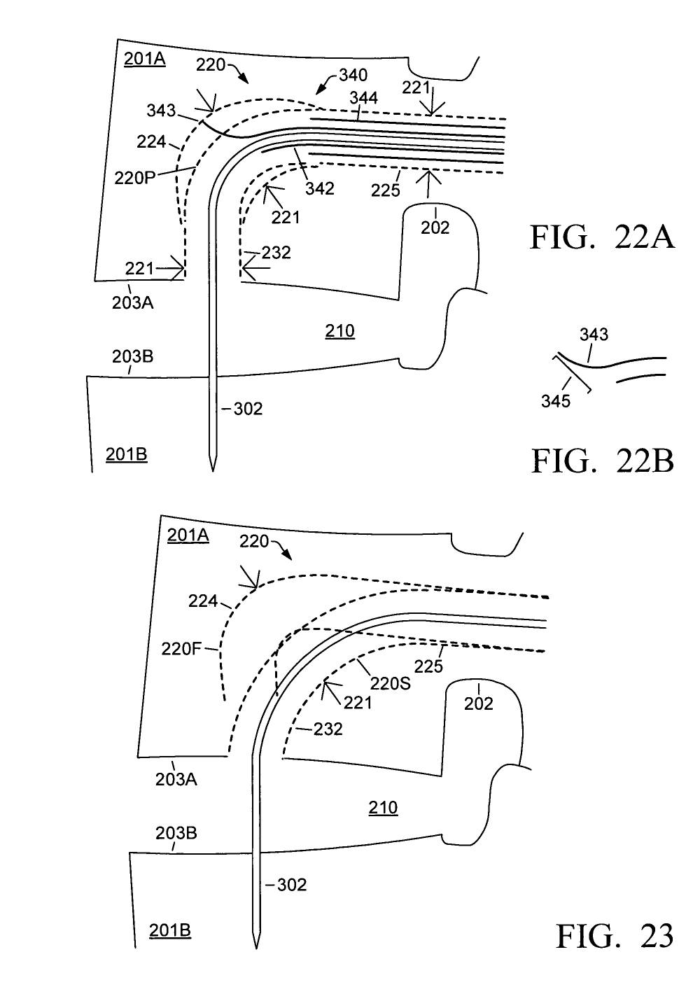

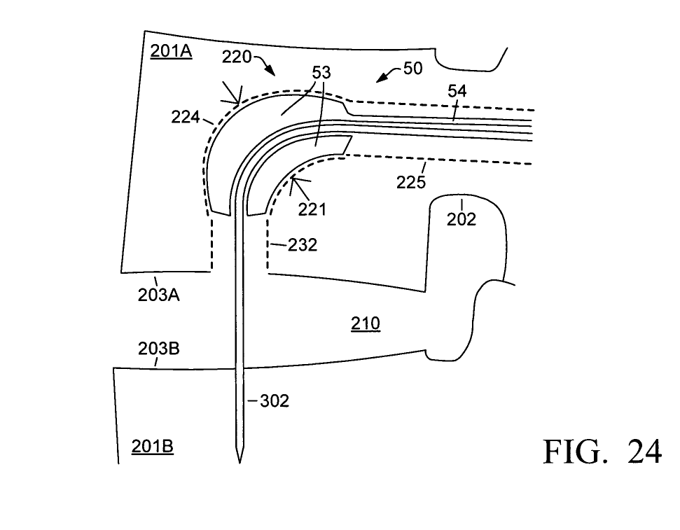

A method of forming a channel 220 with a large channel diameter 221 in the central region 224 is described in connection with FIGS. 22-24.

Guidewire 302 is curved where it passes through the central region 224 of channel 220. This curvature may cause guidewire 302 to bind within a passage 90 in a component, the binding impeding advancing of the component. To reduce binding of guidewire 302, a component may include a flared surface 80 surrounding a portion of a passage 90 or a recess 29. The flared surface 80 accommodates bending of the guidewire 302. For brevity in this paragraph, the term “passage 90” is used to refer to any of the distal component passage 90a, the intermediate component passage 90b, and the proximal component passage 90c.

FIG. 7C is a section view of a proximal component 21 that defines a proximal component recess 29c adjacent the proximal component first end 51, the proximal component recess 29c being coaxial with the proximal component passage 90c. The proximal component 21 has a flared surface 80 surrounding a portion of the proximal component passage 90c or the proximal component recess 29c. The flared surface 80 is curved relative to the proximal component axis 37, the curvature being visible in the longitudinal section view of FIG. 7C. The flared surface 80 may be adjacent the proximal component first end 51, as in the FIG. 7C embodiment, or it may be adjacent the proximal component second end 52. In another embodiment, a flared surface 80 may be included in a proximal component 21 such as that of FIGS. 3-5 which does not include a proximal component recess 29c.

FIG. 7D is a section view of the proximal component 21 of FIG. 7C inserted over a guidewire 302, with a driver 350 for advancing the proximal component 21, the driver including a driver bit 351 and a driver shaft 352. In the FIG. 7D embodiment, driver shaft 352 is a helical coil that is flexible for insertion over guidewire 302. In the embodiments of FIGS. 7B and 7D, the driver bit 351 may have a shape that is, for example, hexagonal in cross-section, with a complementary shape in recess 29a or 29c, in order to drive rotation of the distal component 22 or the proximal component 21 for anchoring in a vertebra 201 using the thread 102.

FIG. 7E is a section view of a distal component 22 that has a flared surface 80 surrounding a portion of the distal component passage 90a, with a driver 350 for advancing the distal component 22, the driver 350 including a driver bit 351 and a driver shaft 352. The flared surface 80 is curved relative to the distal component axis 47, the curvature being visible in the longitudinal section view of FIG. 7E. The flared surface 80 may be adjacent the distal component first end 53 or the distal component second end 54; the FIG. 7E embodiment includes a flared surface 80 adjacent both ends. In another embodiment, a flared surface may be included in a distal component 22 that includes a distal component recess 29a, with the flared surface 80 surrounding a portion of the distal component recess 29a.

FIG. 7F is a section view of a distal component 22 that has a flared surface 80 surrounding a portion of the distal component passage 90a. In the FIG. 7F embodiment, the flared surface 80 is asymmetric relative to the distal component axis 47, so that distal component passage 90a has an oval cross-sectional shape adjacent each of the flared surfaces 80, instead of a circular cross-sectional shape. The overall result is a distal component passage 90a that is generally cylindrical but which bulges to one side adjacent the distal component first end 53 and the distal component second end 54.

In an embodiment that employs a transpedicular posterior approach 240, such as the embodiment of FIG. 7, it may be appropriate to use a channel diameter 221 that is less than or equal to 80 percent of the pedicle height 205 and that is less than or equal to 80 percent of the pedicle width 206. For embodiments that employ an anterior approach 243 or a lateral approach 242, it may be appropriate to use a channel diameter 221 that is not too large relative to the size of the vertebral body 204, in order to maintain the strength of the vertebral body 204. After installation of the device 20, the channel lumen 229 may be filled with bone graft material in order to regenerate new bone, but regeneration of new bone takes time; channel lumen 229 is indicated in FIG. 13.

FIG. 8 is a partial section side view of two vertebrae 201A, 201B in the lumbar region of a human spine, indicating the vertebral body height 219, the pedicle height 205, the channel diameter 221 for the pedicle region 225 of a curved channel 220, and the radius of curvature 223 for the curved region 224 of curved channel 220. FIG. 9 is an axial cephalad view of vertebra L5 in the lumbar region of a human spine, indicating the pedicle width 206 and the channel diameter 221 for the pedicle region 225 of curved channel 220A. The pedicle width 206 and pedicle height 205 are measured at the narrowest part (the isthmus) of pedicle 202. Curved channel 220 has a proximal end 227 and a disc end 226. In FIG. 8 and other figures herein, the dashed line that indicates longitudinal axis 222 is depicted within curved region 224 and is omitted in pedicle region 225.

As described in connection with FIG. 3, the term “curved channel” means that a curved region 224 for curved channel 220 has a radius of curvature 223 that is less than or equal to 100 percent of a vertebral body height 219 for first vertebra 201A. As used herein and in the appended claims, the radius of curvature 223 means the radius of curvature for the longitudinal axis 222 within the curved region 224, the radius of curvature 223 being measured where the curvature is most pronounced. As used herein and in the appended claims, the vertebral body height 219 means the average of the anterior body height and the posterior body height of vertebral body 204, as described in connection with Table 1. In some embodiments, the radius of curvature 223 may be less than or equal to a smaller percent of the vertebral body height 219, such as 95, 90, 85, 80, 75, 70, 65, 60, 55, 50, 45, or 40 percent.

A curved region 224 having a radius of curvature 223 that is less than or equal to 85 percent of vertebral body height 219 is depicted in FIG. 7. For the embodiment of FIG. 13, the radius of curvature 223 for longitudinal axis 222A in curved channel 220 is less than or equal to about 70 percent of vertebral body height 219. Also indicated in FIG. 13 is a second longitudinal axis 222B having a radius of curvature 223 that is less than or equal to 100 percent of vertebral body height 219; in another embodiment, a curved channel 220 may have a curved region 224 with a radius of curvature 223 as for longitudinal axis 222B. For the embodiment of FIG. 14, the radius of curvature 223 for each of curved channels 220A, 220B is less than or equal to about 70 percent of vertebral body height 219. For the embodiment of FIG. 3, the radius of curvature 223 is less than or equal to about 65 percent of the vertebral body height 219. For the curved channel 220 depicted in FIG. 8, the radius of curvature 223 is less than or equal to about 40 percent of the vertebral body height 219.

The dimensions of a vertebra 201, such as pedicle height 205, pedicle width 206, and vertebral body height 219, vary widely between individual humans. Table 1 indicates mean values in millimeters, and ranges for these values, for several dimensions of human lumbar vertebrae L3, L4, and L5. It is understood that the values in Table 1 represent measured values for specific groups of human subjects, and that the actual range of values for dimensions of a vertebra 201 may differ from the range of values indicated in Table 1. The first sacral (S1) vertebra has a vertebral body height 219 that is similar to that of the lumbar vertebrae.

TABLE 1

| | body | pedicle | pedicle | disc |
|---|---|---|---|---|
| | height | width | height | height |
| L3 | 30 | 10 | 15 | 12 |
| | 23-36 | 5-16 | 8-18 | 7-16 |
| L4 | 29 | 13 | 15 | 11 |
| | 22-35 | 9-17 | 9-19 | 5-16 |
| L5 | 28 | 18 | 14 | 11 |
| | 22-35 | 9-29 | 10-19 | 6-16 |

The values for vertebral body height 219 (“body height”) and for disc height are adapted from a journal article by Zhou, S. H., McCarthy, I. D., McGregor, A. H., Coombs, R. R. H., and Hughes, S. P. F., “Geometrical dimensions of the lower lumbar vertebrae—analysis of data from digitised CT images”, Eur. Spine J. 9:242-248, 2000. For the body height for each vertebra L3, L4, and L5, the first line indicates the average of the published mean values for the anterior body height and the posterior body height, and the second line indicates the average of the published range of values for the anterior body height and the posterior body height, each average being rounded to the nearest whole number. The values for pedicle width 206 and pedicle height 205 are adapted from a book entitled “Clinical Biomechanics of the Spine” by White, A. and Panjabi, M., Table 1-6, page 32, J. B. Lippincott Company, 1990. For the pedicle dimensions for each vertebra L3, L4, and L5, the first line indicates the mean value and the second line indicates the range of values. The disc height refers to the height of the spinal disc 210 that is caudal to each vertebra L3, L4, or L5, the disc height being measured at the anterior-posterior midline. For the disc height, the first line indicates the mean value and the second line indicates the range of values, each value being rounded to the nearest whole number.

A normal (undiseased) spine exhibits lordosis in the lumbar region. Thus, the first endplate 203A and the second endplate 203B are slightly angled relative to one another, with a greater spacing between the endplates 203 at the anterior region of spinal disc 210 compared to the spacing at the posterior region of spinal disc 210. When device 20 is installed for treating a spine, device 20 may be installed at a location within endplates 203 that is somewhat anterior to the anterior-posterior midplane of body 204. Installation at an anterior location may assist maintenance or recreation of lordosis.

As depicted in FIG. 9, a plurality of curved channels 220 may be formed in a vertebra 201, so that a plurality of devices 20 may be installed. For example, as depicted in FIG. 9, there may be a pair of curved channels 220A, 220B, with curved channel 220A extending through pedicle 202A and curved channel 220B extending through pedicle 202B. The curvature of curved channels 220A, 220B is evident in side views but not in axial views such as FIG. 9. The plurality of devices 20 may be installed symmetrically with respect to a sagittal plane for the vertebrae 201, as in the embodiment of FIG. 14, or the devices 20 may be installed in some other arrangement.

For a pair of vertebrae 201 that includes a cepahalad vertebra 201 and a caudal vertebra 201, the curved channel 220 can be located in the cephalad vertebra 201 as in FIG. 3 or in the caudal vertebra 201 as in FIG. 8. In other words, first vertebra 201A may be the cephalad vertebra 201 as in FIG. 3, or first vertebra 201A may be the caudal vertebra 201 as in FIG. 8. Device 20 may be used with any vertebra 201 from any region of the spine 200, as long as the dimensions of the vertebra 201 are suitable. For example, the first sacral (S1) vertebra may be the first vertebra 201A or the second vertebra 201B.

In some situations, it may be appropriate to treat multiple levels of a spine using a device 20, thereby stabilizing a first vertebra 201A relative to a second vertebra 201B and also stabilizing the second vertebra 201B relative to a third vertebra 201C. In such embodiments, a device 20 may further comprise a second proximal component 21B and a second distal component 22B. In such embodiments, a first proximal component 21A and a first distal component 22A may be anchored in a first vertebra 201A and a second vertebra 201B, respectively, while a second proximal component 21B and a second distal component 22B may be anchored in the second vertebra 201B and a third vertebra 201C, respectively. In one embodiment, the device 20 may be installed as follows: (1) advancing the second distal component 22B and the second proximal component 21B through the curved channel 220 within first vertebra 201A and through a channel extension within second vertebra 201B; and (2) advancing the first distal component 22A and the first proximal component 21A through the curved channel 220. In another embodiment, a second curved channel 220B may be formed in the second vertebra 201B or in the third vertebra 201C, and the second distal component 22B and the second proximal component 21B may be advanced through the second curved channel 220B.

Much of the information described in connection with FIGS. 1-9 applies generally to other embodiments. Thus, this general information is not repeated in the description of each embodiment.

FIG. 10 is a partial section side view of two vertebrae 201A, 201B and a device 20 for treating a spine, the device 20 comprising a proximal component 21 that is anchored in a first vertebra 201A and a distal component 22 that is anchored in a second vertebra 201B, in accordance with an embodiment. FIG. 11 is a section view of the device 20 of FIG. 10, with the plane of section taken along line A-A′ of FIG. 10. FIG. 12 is a section view of the individual components of the device 20 of FIG. 10, with the plane of section for each component taken along line A-A′ of FIG. 10. A proximal component axis 37 and a distal component axis 47 are indicated, and are discussed in connection with FIG. 6.

The embodiment of FIGS. 10-12 is very similar to the embodiment of FIGS. 3-5, with several differences. A first difference between the embodiment of FIGS. 10-12 and that of FIGS. 3-5 is that the FIG. 10-12 embodiment does not comprise an intermediate component 60. Proximal component 21 releasably engages distal component 22, with a first end 53 for distal component 22 seated in a recess 29 at a second end 52 for proximal component 21. A second difference is the location of the recess 29; in the embodiment of FIGS. 10-12 the recess 29 is in proximal component 21, not in distal component 22 as in the embodiment of FIGS. 3-5. In another embodiment, a recess 29 at an end of a first component may be threaded, with complementary threading on an end of a second component that releasably engages the threaded recess of the first component. A threaded engagement may be most appropriate in a relatively large device 20 embodiment intended for installation in a relatively large vertebra 201. A relatively large device 20 having relatively large component diameters may provide greater space to include threading, compared to a relatively small device 20.

A third difference is that the distal component length 43 for the FIG. 10-12 embodiment is significantly larger than the distal component length 43 for the FIG. 3-5 embodiment. The overall length of the device 20 is similar for the FIG. 10-12 embodiment and the FIG. 3-5 embodiment. The larger distal component length 43 in the FIG. 10-12 embodiment substitutes for the intermediate component 60 in the FIG. 3-5 embodiment.

A component may be uniform in diameter or may be narrowed. In a narrowed component, the transverse dimension for the component in smaller in some portions of the component than in other portions. Narrowing of a component may facilitate passage of the component through curved channel 220. As used herein and in the appended claims, the terms narrowing and tapering mean large scale differences in transverse dimension within a component, and not local variations in transverse dimension that result from threading or other anchor means.

In a unidirectional type of narrowing, the transverse dimension for the component is largest at or near one end of the component, and the transverse dimension decreases toward the other end of the component. Unidirectional narrowing results in a component that has a shape that is conical or trapezoidal or bullet-like when viewed in longitudinal section. If the narrowing is gradual, then the unidirectional narrowing is a unidirectional taper. The distal component 22 in the embodiment of FIGS. 3-5 has a bullet-like shape, with a rounded second end 54. The proximal component 21 in the embodiment of FIGS. 3-5 or the embodiment of FIGS. 10-12 has a roughly trapezoidal shape, with a blunt second end 52.

In a bidirectional type of narrowing, the transverse dimension for the component is largest in the central region of the component, and the transverse dimension decreases towards both ends of the component. Bidirectional narrowing results in a component that has a shape that is a diamond or oval or a double trapezoid when viewed in longitudinal section. If the narrowing is gradual, then the bidirectional narrowing is a bidirectional taper. This distal component 22 in the embodiment of FIGS. 10-12 has an oval or double trapezoid shape.

A component that is not narrowed has the same transverse dimension from one end to the other end, and has a shape that is roughly rectangular when viewed in longitudinal section. The distal component 22A depicted in FIG. 7, which is uniform in diameter except for small variations caused by thread 102, is an example of a not narrowed component. The not narrowed distal components 22A and 22B of FIG. 7 each have a roughly rectangular shape, except for the variations caused by thread 102.

Narrowing may be gradual (a taper) or abrupt (a step). Narrowing of a component may be gradual, so that the component or a portion of the component is tapered. The distal component 22 of FIGS. 3-5 and the proximal component 21 of FIGS. 10-12 are examples of tapered components. The slope of tapering may change from one region of a component to another. The proximal component 21 of FIGS. 3-5 has a gentle slope of tapering in the region that includes first end 51 and thread 102, and has a steeper slope of tapering in a region near second end 52. Narrowing may be abrupt, with a “step” or relatively steep change in diameter. The intermediate component 60 in the embodiment of FIGS. 3-5 has an abrupt narrowing or step.

Tapering may facilitate the engagement and seating of a component with another component during installation of the device 20. In an embodiment with tapered seating, a tapered region on a first component is seated in a tapered recess on a second component. As described for the embodiment of FIGS. 10-12, for example, proximal component 21 releasably engages distal component 22, with a first end 53 for distal component 22 seated in a recess 29 at a second end 52 for proximal component 21. Distal component 22 includes a tapered region 44, and the recess 29 of proximal component 21 has an internal taper that is roughly complementary to the external taper of tapered region 44. As described in connection with FIG. 6, during installation of the device 20 the orientation of proximal component 21 changes while the proximal component 21 passes through the curved channel 220. As proximal component 21 advances so that it is adjacent to distal component 22, proximal component axis 37 may not yet be exactly colinear with distal component axis 47. If recess 29 in proximal component 21 were straight sided instead of tapered, and if the region near first end 53 of distal component 22 were straight sided instead of tapered, it might be difficult for first end 53 to seat within recess 29 when the components are not yet colinear. When first end 53 and recess 29 are tapered, the tapering facilitates seating in spite of the components not yet being colinear.

FIG. 13 is a partial section side view of two vertebrae 201A, 201B and a device 20 for treating a spine, the device 20 comprising a proximal component 21 that is anchored in a first vertebra 201A and a distal component 22 that is anchored in a second vertebra 201B, in accordance with an embodiment. The embodiment of FIG. 13 employs an anterior approach 243. For the embodiment of FIG. 13, the radius of curvature 223 for longitudinal axis 222A in curved channel 220 is less than or equal to about 70 percent of vertebral body height 219, as described in connection with FIG. 8. Also indicated in FIG. 13 is a second longitudinal axis 222B having a radius of curvature 223 that is less than or equal to 100 percent of vertebral body height 219. In another embodiment, a curved channel 220 may have a curved region 224 with a radius of curvature 223 as for longitudinal axis 222B.

In the embodiment of FIG. 13, the intermediate component 60 comprises a plurality of intermediate components 60. A plurality of intermediate components 60 may facilitate spanning the distance between the vertebrae 201A and 201B which is occupied by the spinal disc 210. In the embodiment of FIG. 13, the disc height for the spinal disc 210 is about one-third of the vertebral body height 219. This ratio of heights is consistent with mean values for disc height and for vertebral body height 219, as indicated in Table 1.

In the embodiment of FIG. 13, the upper or superior one of the plurality of intermediate components 60 is positioned partially within the spinal disc 210 and partially within the first vertebra 201A. In another embodiment, an intermediate component 60 may include means for anchoring in a vertebra 201, thereby augmenting the anchoring provided by first means for anchoring 23 and second means for anchoring 24.

Installation of device 20 may include introducing bone graft material or another bone growth substrate into channel lumen 229 to promote regeneration of bone in the region of curved channel 220 that extends from channel proximal end 227 to device 20, thereby strengthening first vertebra 201A. Channel proximal end 227 may be sealed with a cap 94 which may be secured by various means. In another embodiment, channel proximal end 227 may be sealed with a screw driven into body 204.

FIG. 14 is a partial section anterior view of the bodies 204 of two vertebrae 201A, 201B and plural devices 20 for treating a spine, each device 20 comprising a proximal component 21 that is anchored in a first vertebra 201A and a distal component 22 that is anchored in a second vertebra 201B, in accordance with an embodiment. The embodiment of FIG. 14 employs a lateral approach 242. Plural devices 20 are installed to stabilize the vertebrae 201A, 201B, with the arrangement of devices 20 being symmetrical about a sagittal plane. One of the plurality of devices 20 is installed by passing components through curved channel 220A, and the other device 20 is installed by passing components through curved channel 220B.

In the embodiments depicted herein, each individual component is a single piece. In other embodiments, a component may comprise plural pieces. For example, a component may comprise an inner portion and an outer portion, or a component may comprise several portions that are positioned side by side. A component with several portions may facilitate expansion anchoring of the component and may also serve to distract a pair of vertebrae 201A, 201B.

In one embodiment, a component may comprise an outer portion or fitting that is anchored in a vertebra 201 and an inner portion or forcing member that is capable of moving relative to the outer portion or fitting. In such an embodiment, the forcing member may exert force in an outward or lateral direction and may also exert force in the direction of the component axis. The forcing member or the outer portion may be tapered so that axial displacement of the forcing member causes outward or lateral force on the outer portion or fitting. There may be internal threading on the outer portion that engages external threading on the forcing member.

In such an embodiment, first means for anchoring 23 or second means for anchoring 24 may include ridges or other protrusions on the external surface of the outer member or fitting for anchoring by expansion. The outer member or fitting may be a cylinder or another hollow form with a longitudinal slit, made of a material that is sufficiently flexible to allow outward expansion of the outer portion in response to the forcing member. In another embodiment, the outer portion or fitting may be a set of elongate pieces, linked at their first ends or linked at their second ends, with ridges or other means for anchoring on the external surface of the elongate pieces.

For axial displacement of the forcing member, a tool has an outer element that pulls on the first end of the component outer portion while an inner element for the tool pushes or rotates the first end of the forcing member. In one embodiment, the proximal component 21 includes an outer portion and an inner portion or forcing member as described. Axial displacement of the forcing member may be used to exert force on an intermediate component 60 or on a distal component 22, thereby causing distraction of a pair of vertebrae 201A, 201B.

As used herein and in the appended claims, the term “thread” 102 means a helical or spiral ridge on a screw, nut, or bolt, or on a cylindrical component such as the proximal component 21 or the distal component 22 in the embodiment of FIG. 3. As used herein and in the appended claims, the term “ridge” 103 means an elongate protrusion on the surface of a component; the component having the ridge may or may not have a cylindrical shape; the surface of the component may be flat or curved.

Components and portions of components of device 20 may be made from various materials known to be suitable for use in medical devices and in particular for use in devices for treating bones including the spine. Such materials include metals such as titanium or stainless steel or cobalt. Such materials include metal alloys such as titanium alloys, including alloys of titanium and stainless steel, and “shape memory” alloys such as nitinol. Such materials include polymers such as polyetheretherketone (“PEEK”). Such materials may also include ceramics such as ceramic materials used in hip implants.

Table 2 indicates a method for treating a spine, the method comprising a series of steps that are listed in Table 2, in accordance with an embodiment.

TABLE 2
A method for treating a spine, the spine including a first vertebra 201A and a second vertebra
201B, the first vertebra 201A having a first endplate 203A that is adjacent a spinal disc 210, the
second vertebra 201B having a second endplate 203B that is adjacent the spinal disc 210, the first
vertebra 201A having a first pedicle 202, the method comprising:

(a) providing a proximal component 21, a distal component 22, and a guidewire 302, wherein
the proximal component 21 includes first means for anchoring 23 in the first vertebra 201A and the
proximal component 21 has a proximal component axis 37 that is substantially straight, wherein the
distal component 22 includes second means for anchoring 24 in the second vertebra 201B and the
distal component 22 has a distal component axis 47 that is substantially straight;
(b) forming a curved channel 220 that extends through the first vertebra 201A from the first
pedicle 202 to the first endplate 203A, the curved channel 220 having a radius of curvature 223 that
is less than or equal to 100 percent of a vertebral body height 219 for the first vertebra 201A,
wherein the guidewire 302 extends through the curved channel 220 and into the second vertebra
201B;
(c) advancing the distal component 22 over the guidewire 302 through the curved channel
220 to a distal component position for the distal component 22, wherein the distal component
position is entirely within the second vertebra 201B or partially within the second vertebra 201B and
partially within the spinal disc 210;
(d) anchoring the distal component 22 at the second vertebra 201B;
(e) advancing the proximal component 21 over the guidewire 302 through the curved channel
220 to a proximal component position for the proximal component 21, wherein the proximal
component position is entirely within the first vertebra 201A or partially within the first vertebra
201A and partially within the spinal disc 210; wherein the proximal component axis 37 has an
orientation relative to the distal component axis 47; wherein the orientation changes by at least 40
degrees while the proximal component 21 passes through the curved channel 220; and
(f) anchoring the proximal component 21 at the first vertebra 201A, wherein the proximal
component axis 37 is subtantially perpendicular to the first endplate 203A.

In another embodiment, the method further comprises providing an intermediate component 60; and prior to step (e), advancing the intermediate component 60 over the guidewire 302 through the curved channel 220 to an intermediate component position for the intermediate component 60, wherein the intermediate component 60 releasably engages the distal component 22.

In another embodiment, the method further comprises distracting the first vertebra 201A and the second vertebra 201B. In another embodiment, the method further comprises inducing fusion of the first vertebra 201A and the second vertebra 201B. In another embodiment, the inducing fusion comprises preparing the spinal disc 210 and introducing a bone growth substrate within the spinal disc 210. The bone growth substrate may be bone graft or another substrate that promotes growth of bone. In another embodiment, the method further comprises introducing a bone growth substrate within the channel lumen 229.

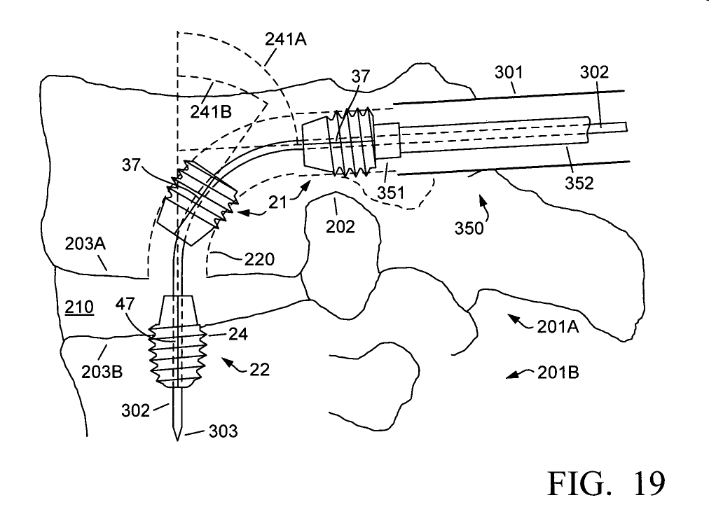

FIGS. 15-19 depict a first vertebra 201A and a second vertebra 201B at various stages during performance of a method for treating a spine, in accordance with an embodiment. The method embodiment of FIGS. 15-19 employs a device 20 that is similar to the device 20 embodiment of FIGS. 10-12, the device 20 having two components each of which is a single piece. FIGS. 15-17 depict details of the forming of a curved channel 220 (step b). FIG. 18 depicts the advancing of the distal component 22 through the curved channel 220 (step c). FIG. 19 depicts the advancing of the proximal component 21 through the curved channel 220 (step e). In the embodiment depicted in FIGS. 15-19, the first vertebra 201A is the cephalad vertebra 201 of the pair of vertebra 201, and the curved channel 220 extends in a caudal direction. In another embodiment, the first vertebra 201A may be the caudal vertebra 201 of the pair, in which case the curved channel 220 would extend in a cephalad direction.

The method embodiment depicted in FIGS. 15-19 is performed using a percutaneous transpedicular posterior approach 240. Other embodiments may use an anterior approach 243 or a lateral approach 242. In the transpedicular posterior approach 240 used in the embodiment of FIGS. 15-19, a standard bone drill may be used to drill through the first pedicle 202 to the body 204. This initial channel segment corresponds to the pedicle region 225 of what will eventually become curved channel 220. The channel diameter 221 for the initial channel segment is selected in relation to the dimensions for the first vertebra 201A, as described herein. A cannula 301 may be inserted into the initial channel segment.

In the embodiment of FIGS. 15-19, a narrow curved pilot channel is formed using a steerable channel forming tool, which in this embodiment is a steerable drilling device 330. The narrow curved pilot channel is a precursor to the curved region 224 of the curved channel 220. For embodiments that use an anterior approach 243 or a lateral approach 242, the curved channel 220 includes a curved region 224 but no pedicle region 225, and the curved region 224 begins at a hole drilled in the body wall 230. In the depicted embodiment, the narrow curved pilot channel extends in an anterior and caudal direction, so that upon completion of the forming of the narrow curved pilot channel the axis at the tip of the steerable channel forming tool is substantially perpendicular to the first endplate 203A, as depicted in FIG. 15. As used herein and in the appended claims, the term “substantially perpendicular” means an angle 228 having a value between 75 degrees and 105 degrees. In other words, angle 228 has a value that is greater than or equal to 75 degrees and less than or equal to 105 degrees. FIG. 15 includes three dashed lines labelled A, B, and C that intersect first endplate 203A at three different angles 228: line A intersects at 75 degrees, line B intersects at 90 degrees, and line C intersects at 105 degrees.

The narrow curved pilot channel may stop short of the first endplate 203A, as depicted in FIG. 15, or may penetrate the first endplate 203A. The steerable channel forming tool is steered so that the resulting narrow curved pilot channel has an appropriate radius of curvature 223 in relation to the vertebral body height 219 for first vertebra 201A and so that the narrow curved pilot channel is substantially perpendicular to the first endplate 203A, as described herein.

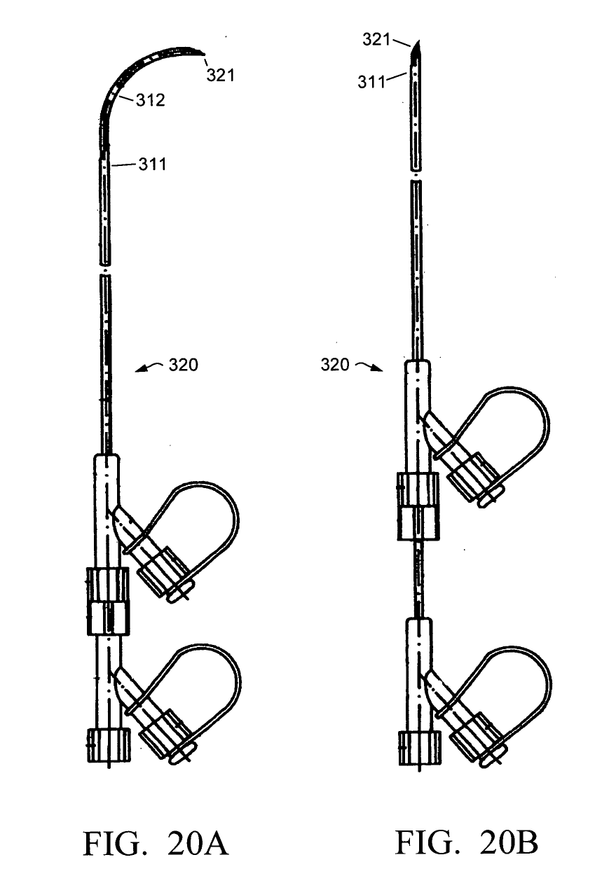

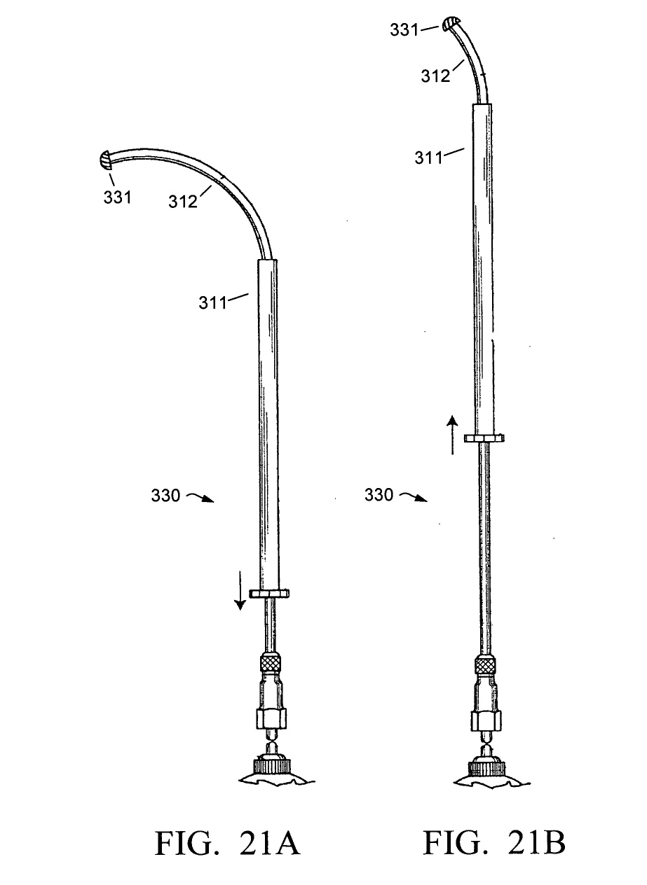

Various steerable channel forming tools may be used to form the narrow curved pilot channel. FIGS. 20 and 21 depict two examples of steerable channel forming tools. The tools of FIGS. 20 and 21 each include an outer tube 311 that is relatively rigid and an elastic precurved tube 312 disposed within the outer tube 311. The elastic precurved tube 312 may be advanced and retracted relative to the outer tube 311 in a telescopic manner. Retraction of the elastic precurved tube 312 within outer tube 311 causes straightening of the elastic precurved tube 312. Advancing of the elastic precurved tube 312 so that it extends beyond the outer tube 311 allows the elastic precurved tube 312 to regain its curvature, causing the tip of the elastic precurved tube 312 to point in a direction that is not aligned with the axis of the outer tube 311, thereby enabling the forming of a narrow curved pilot channel.

The steerable channel forming tool depicted in FIGS. 20A-20B is a steerable needle 320 having a bevelled tip 321 at the end of the elastic precurved tube 312. FIGS. 20A and 20B are adapted from FIGS. 6 and 7 of U.S. Pat. No. 6,572,593 issued to Daum. The steerable channel forming tool depicted in FIGS. 21A-21B is a steerable drilling device 330 having a drill bit 331 at the end of the elastic precurved tube 312. FIGS. 21A and 21B are adapted from FIGS. 7 and 8 of U.S. Pat. No. 6,740,090 issued to Cragg. A steerable drilling device 330 very similar to that of the U.S. Pat. No. 6,740,090 patent is described in detail in U.S. Pat. No. 7,241,297 issued to Shaolian. Another type of steerable channel forming tool is a tension wire drill such as that depicted in FIGS. 19 and 20 of the U.S. Pat. No. 6,740,090 patent.

In the embodiment of FIGS. 15-16, the steerable channel forming tool is a steerable drilling device 330 having a drill bit 331 and a flexible drive shaft 332. Flexible drive shaft 332 is a hollow tubular drive shaft capable of receiving a guide wire 302. Drill bit 331 similarly has a passage for a guide wire 302. After the forming of the narrow curved pilot channel, a guide wire 302 is introduced into the lumen of flexible drive shaft 332 and the guide wire 302 is advanced so that it extends through and beyond drill bit 331. As noted previously, the axis at the tip of the steerable channel forming tool is substantially perpendicular to the first endplate 203A. The guide wire 302, where it emerges from the elastic precurved tube 312, is substantially perpendicular to the first endplate 203A.

The guide wire 302 has a sharp tip 303. As depicted in FIG. 16, the guide wire 302 is advanced so that the tip 303 penetrates the first endplate 203A, the spinal disc 210, and the second endplate 203B, and then continues further into the body 204 of second vertebra 201B. The steerable drilling device 330 is withdrawn without disturbing the guide wire 302, which remains in place with the guidewire tip 303 poking into second vertebra 203B.

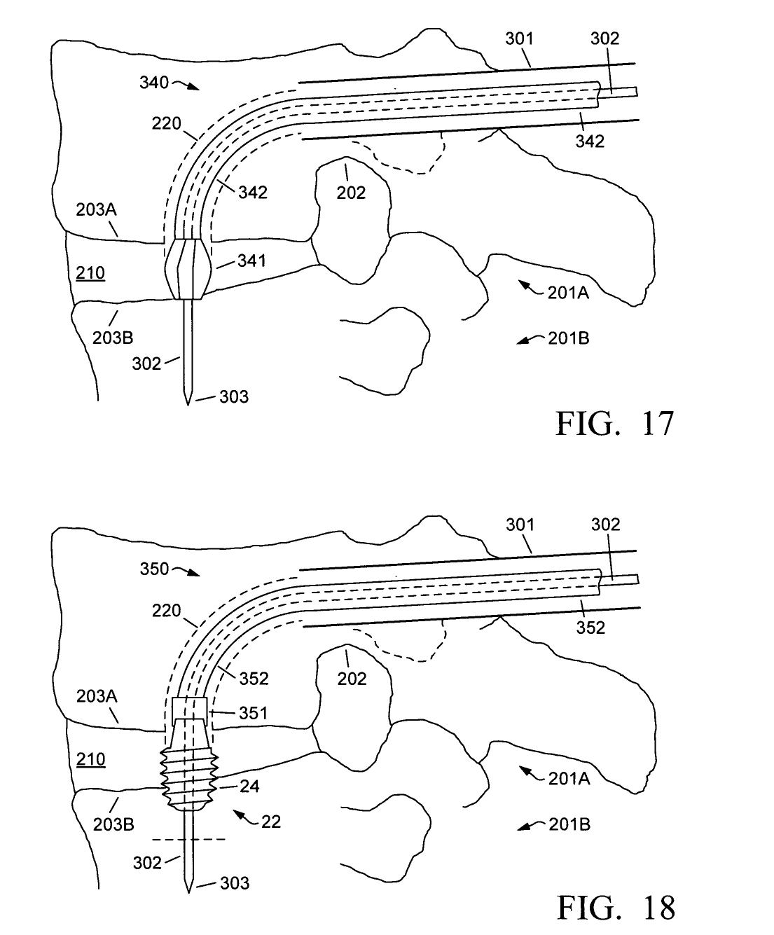

A flexible drill 340 is then introduced into cannula 301 over guide wire 302. The flexible drill 340 has a hollow flexible drive shaft 342 and a cutting head 341 that has a passage for the guide wire 302. The flexible drill 340 may be used to enlarge the narrow curved pilot channel within first vertebra 201A and to extend the channel through the first endplate 203A, the spinal disc 210, and the second endplate 203B. FIG. 17 depicts the stage when the cutting head 341 is advancing through the spinal disc 210 into the second endplate 203B. The enlarged channel includes a curved channel 220 as depicted in other Figures herein. The flexible drill 340 may continue through the spinal disc 210 to form a channel extension 245 in second vertebra 201B, as depicted in FIG. 7B. The flexible drill 340 is withdrawn without disturbing the guide wire 302, which remains in place with the guidewire tip 303 poking into second vertebra 203B.

FIG. 18 depicts the advancing of the distal component 22 through the curved channel 220 (step c). Distal component 22 is inserted into cannula 301 over guide wire 302 together with a flexible driver 350. Flexible driver 350 engages distal component 22 at a slot or recess at or adjacent first end 53 of distal component 22; other flexible drivers are described in connection with FIGS. 7B-7F. Flexible driver 350 has a hollow flexible drive shaft 352 and a driver tip 351 with a passage for guide wire 302. In another embodiment, driver tip 351 may include a socket that engages the distal component tapered region 44, as depicted in FIG. 7E. Distal component 22 and driver tip 351 advance together through curved channel 220 until distal component 22 reaches the second endplate 203B. Rotation of flexible driver 350 causes rotation of distal component 22 so that thread 102 (second means for anchoring 24 ) engages second endplate 203B and/or cancellous bone adjacent second endplate 203B and distal component 22 screws into second vertebra 201B, resulting in anchoring of distal component 22 in second vertebra 201B (step d). Distal component 22 may be advanced until, for example, second end 54 is at the horizontal dashed line, depicted in FIG. 18, that crosses guide wire 302 near tip 303. Flexible driver 350 is withdrawn without disturbing the guide wire 302, which remains in place with the guide wire 302 extending through distal component 22 into second vertebra 203B.

FIG. 19 depicts the advancing of the proximal component 21 through the curved channel 220 (step e). In the embodiment of FIG. 19, the curved channel 220 has been enlarged in the curved region 224, after the anchoring of distal component 22 at second vertebra 201B and before the advancing of proximal component 21. In addition, a cannula 301 having a slightly larger diameter has been inserted in place of the cannula 301 depicted in FIGS. 15-18. Proximal component 21 is depicted at a first location at a first time and is also depicted at a second location at a second time. The orientation of proximal component 21 relative to distal component 22 during installation of device 20 is discussed in connection with FIGS. 3-6.

As depicted in FIG. 19, proximal component 21 is inserted into cannula 301 over guide wire 302 together with flexible driver 350. Proximal component 21 is advanced as described for distal component 22 and is anchored at first vertebra 201A by rotation, resulting in anchoring of proximal component 21 at first vertebra 201A (step f). Proximal component 21 is advanced far enough so that it engages distal component 22. In another embodiment described in the following paragraph, proximal component 21 releasably engages an intermediate component 60 instead of engaging distal component 22. Optionally, proximal component 21 may be advanced further in order to distract vertebrae 201A and 201B. In other words, the method may further comprise distracting the first vertebra 201A and the second vertebra 201B. Flexible driver 350 is withdrawn, and guide wire 302 and cannula 301 are withdrawn.

In another embodiment, the method further comprises providing an intermediate component 60 and advancing the intermediate component 60 through the curved channel 220 to an intermediate component position for the intermediate component 60, wherein the intermediate component 60 releasably engages the distal component 22. The intermediate component 60 is advanced through the curved channel 220 after the anchoring of the distal component 22 at the second vertebra 201B (step d), and prior to the advancing of the proximal component 21 through the curved channel 220 (step e). The proximal component 21 is advanced far enough so that it releasably engages the intermediate component 60. FIGS. 3 and 4 depict the embodiment after all three components have been advanced through the curved channel 220, with the intermediate component 60 releasably engaging the distal component 22, and the proximal component 21 releasably engaging the intermediate component 60.

In another embodiment, the method further comprises inducing fusion of the first vertebra 201A and the second vertebra 201B. In another embodiment, the inducing fusion comprises preparing the spinal disc 210 and introducing a bone growth substrate within the spinal disc 210. The preparing of the spinal disc 210 may include removing some or all of the spinal disc 210, and the preparing may include removing some or all of the cartilage that is external to first endplate 203A or second endplate 203B. The removing of some or all of the spinal disc 210 or the cartilage may employ a directed jet of water as in cutting devices supplied by Hydrocision Corporation of Massachusetts, US. The removing of some or all of the spinal disc 210 or the cartilage may employ a cutting device or an enucleation device such as those depicted in FIGS. 31-36 of U.S. Pat. No. 7,318,826 issued to Teitelbaum.

The bone growth substrate may be bone graft or another substrate that promotes growth of bone. The introducing of the bone growth substrate may employ a flexible tube that is inserted into cannula 301 over guide wire 302 and that extends through part or all of curved channel 220. The introducing of the bone growth substrate may be performed before the advancing of the distal component 22 (step c) or may be performed later. In one embodiment, the introducing of the bone growth substrate may be performed through a separate channel within first vertebra 201A or a separate channel within second vertebra 201B. For example, the separate channel may be a second curved channel 220B within first vertebra 201A, the second curved channel 220B being intended also for installation of a second device 20B. The separate channel may have a diameter that is different from the channel diameter 221 for the curved channel 220. In an embodiment that uses a separate channel for introducing the bone growth substrate, the introducing of the bone growth substrate may be performed after completion of installation of device 20.

As described herein in connection with FIG. 7B, a channel 220 may have a variable diameter. In one embodiment, the channel diameter 221 for the central region 224 is greater than the channel diameter 221 for the pedicle region 225 and the channel diameter 221 for the central region 224 is greater than the channel diameter 221 for the endplate region 232.

Table 3 indicates a method for treating a spine, the method comprising a set of steps (a)-(d) that are listed in Table 3, in accordance with an embodiment.

TABLE 3
A method for treating a spine, the spine including a first vertebra 201A and a second vertebra
201B, the first vertebra 201A having a first endplate 203A that is adjacent a spinal disc 210, the
second vertebra 201B having a second endplate 203B that is adjacent the spinal disc 210, the first
vertebra 201A having a body 204 and a pedicle 202, the method comprising:

(a) forming a channel 220 that extends through the first vertebra 201A, wherein the channel
220 extends through the pedicle 202 and through the first endplate 203A, the channel 210 having a
channel diameter 221, the channel 220 having a pedicle region 225, a central region 224, and an
endplate region 232, wherein the channel diameter 221 for the central region 224 is greater than the
channel diameter 221 for the pedicle region 225 and the channel diameter 221 for the central region
224 is greater than the channel diameter 221 for the endplate region 232;
(b) providing an implant, the implant having an implant diameter, wherein the implant
diameter is configured to permit passage of the implant through the pedicle region 225 and through
the endplate region 232;
(c) introducing the implant into the pedicle region 225; and
(d) advancing the implant through the channel 220, wherein at least a portion of the implant
advances at least to the first endplate 203A.

The channel forming step (step a) may be performed as described in connection with FIGS. 22-24 and FIGS. 15-19 and FIGS. 20-21. FIGS. 15-19 depict general aspects of forming a channel 220, and FIGS. 20-21 depict steerable tools that may be used in forming a channel 220. FIGS. 22-24 depict method embodiments for forming a variable diameter channel 220. The methods and tools described in connection with FIGS. 15-19 and FIGS. 20-21 may be used, for example, to form predecessor channels in the FIG. 22-24 embodiments.

With respect to the providing step (step b), a variable diameter channel 220 may be used with many types of implant. The provided implant may be a component of a device 20 that comprises several components, such as a proximal component 21 or a distal component 22 as described herein. In such an embodiment, steps (c) and (d) may be repeated for each of the implants or components. In another embodiment, the implant may be a single piece that is installed without any other cooperating component. As indicated in step (b), the provided implant has an implant diameter that is configured to permit passage of the implant through the pedicle region 225 and through the endplate region 232.

With respect to step (c), the implant may be introduced into the pedicle region 225 using a posterior approach 240 as depicted in FIGS. 1-2. In one embodiment, the surgical approach is percutaneous and employs a cannula 301 such as that depicted in FIGS. 15-19.

With respect to step (d), the implant may be advanced through the channel 220 using a flexible driver 350 and a guidewire 302, as described herein in connection with various Figures, or the implant may be advanced using a steerable driver tool such as a driver tool that is steered using a tension wire.

In another embodiment, the method further comprises installing the implant, wherein the installing comprises positioning the implant at least partially within the spinal disc 210 or at least partially within the first vertebra 201A or at least partially within the second vertebra 201B. For example, the implant may be positioned entirely within the spinal disc 210, as is typical for the positioning of a spinal interbody spacer implant. In another example, the implant may be positioned partially within the spinal disc 210 and partially within the second vertebra 201B, as depicted for distal component 22 in FIGS. 3 and 6 herein. In another example, the implant may be positioned partially or entirely within first vertebra 201A, as depicted for proximal component 21 in FIGS. 3 and 10 herein.

FIGS. 22-24 depict method embodiments for forming a variable diameter channel 220, as described in the following paragraphs.

In another embodiment, the channel forming step (step a) comprises creating a predecessor channel that extends through the pedicle 202 and through the first endplate 203A, wherein the predecessor channel is coaxial with the channel 220 in at least a portion of the pedicle region 225 and the predecessor channel is coaxial with the channel 220 in at least a portion of the endplate region 232; and enlarging the central region 224 for the predecessor channel, wherein the enlarging causes the channel diameter 221 for the central region 224 to be greater than the channel diameter 221 for the pedicle region 225 and the enlarging causes the channel diameter 221 for the central region 224 to be greater than the channel diameter 221 for the endplate region 232. The embodiment described in the previous sentence includes embodiments such as those depicted in FIGS. 22 and 24, which are described in subsequent paragraphs.

In an embodiment that is depicted in FIGS. 22A and 22B, the enlarging step comprises cutting or abrading the body 204 where it surrounds the central region 224 of the predecessor channel 220P using a drill, the drill comprising a steerable drill or a flexible drill 340, the drill comprising a retractable cutting head 343 and a sheath 344, the retractable cutting head 343 being capable of retracting within the sheath 344, the sheath 344 dimensioned to be insertable within the predecessor channel 220P, the retractable cutting head 343 capable of emerging from a distal end of the sheath 344, wherein a cutting head radius 345 for the emerged retractable cutting head 343 is greater than half of the channel diameter 221 for the pedicle region 225.

In another embodiment, the enlarging step comprises advancing a dilator in the predecessor channel to a position within the central region 224, and dilating the dilator for displacing cancellous bone of the body 204 that surrounds the central region 224 of the predecessor channel. In one embodiment that is depicted in FIG. 24, the dilator comprises a balloon 53 and an inflation line 54 that is connected to the balloon 53, and the dilating comprises inflating the balloon 53. In another embodiment, the dilator may comprise a wedge. For example, the dilator may comprise a flexible sleeve and a wedge that is insertable within a narrow lumen of the flexible sleeve, the inserting of the wedge forcing the sleeve outward to displace cancellous bone.

In another embodiment that is depicted in FIG. 23, the channel forming step (step a) comprises creating a first predecessor channel 220F and a second predecessor channel 220S, wherein the second predecessor channel 220S diverges from the first predecessor channel 220F in at least a portion of the central region 224. In the embodiment of FIG. 23, the central region 224 has an oval cross-section.

Although we have described in detail various embodiments, other embodiments and modifications will be apparent to those of skill in the art in light of this text and accompanying drawings. The following claims are intended to include all such embodiments, modifications and equivalents.

What is claimed is:

1. A device for treating a spine, the spine including a first vertebra and a second vertebra, the first vertebra having a first endplate that is adjacent a spinal disc, the second vertebra having a second endplate that is adjacent the spinal disc, the first vertebra having a first pedicle, wherein a curved channel extends through the first vertebra from the first pedicle to the first endplate, the curved channel having a radius of curvature that is less than or equal to 100 percent of a vertebral body height for the first vertebra, the device comprising:
a proximal component, wherein the proximal component includes first means for anchoring in the first vertebra; wherein the proximal component has a proximal component position; wherein the proximal component position is entirely within the first vertebra or partially within the first vertebra and partially within the spinal disc;
wherein the proximal component has a proximal component axis; wherein the proximal component axis is substantially straight; wherein the proximal component axis is substantially perpendicular to the first endplate; and
a distal component, wherein the distal component includes second means for anchoring in the second vertebra; wherein the distal component has a distal component position; wherein the distal component position is entirely within the second vertebra or partially within the second vertebra and partially within the spinal disc;
wherein the distal component has a distal component axis; wherein the distal component axis is substantially straight;
wherein the proximal component axis has an orientation relative to the distal component axis; and
wherein during installation of the device the orientation changes by at least 40 degrees while the proximal component passes through the curved channel.

2. The device of claim 1,
wherein the proximal component releasably engages the distal component.

3. The device of claim 1,
further comprising an intermediate component,
wherein the proximal component releasably engages the intermediate component and the intermediate component releasably engages the distal component.

4. The device of claim 3,
wherein the intermediate component comprises a plurality of intermediate components.

5. A device for treating a spine, the spine including a first vertebra and a second vertebra, the first vertebra having a first endplate that is adjacent a spinal disc, the second vertebra having a second endplate that is adjacent the spinal disc, the device comprising:
a proximal component, the proximal component having a proximal component first end and a proximal component second end, the proximal component comprising a proximal component tapered region adjacent the proximal component second end and a first thread for anchoring in the first vertebra, the proximal component defining a proximal component passage that is capable of receiving a guidewire, the proximal component passage extending from the proximal component first end to the proximal component second end;
an intermediate component, the intermediate component having an intermediate component first end and an intermediate component second end, the intermediate component comprising an intermediate component tapered region adjacent the intermediate component second end, the intermediate component defining an intermediate component passage that is capable of receiving the guidewire, the intermediate component passage extending from the intermediate component first end to the intermediate component second end, the intermediate component defining an intermediate component recess adjacent the intermediate component first end, the intermediate component recess being coaxial with the intermediate component passage; and
a distal component, the distal component having a distal component first end and a distal component second end, the distal component comprising a second thread for anchoring in the second vertebra, the distal component defining a distal component passage that is capable of receiving the guidewire, the distal component passage extending from the distal component first end to the distal component second end, the distal component defining a distal component recess adjacent the distal component first end, the distal component recess being coaxial with the distal component passage;
wherein the intermediate component tapered region is capable of being seated within the distal component recess, and wherein the proximal component tapered region is capable of being seated within the intermediate component recess.

6. The device of claim 5,
wherein the distal component has a flared surface surrounding a portion of the distal component passage or the distal component recess, the flared surface being curved relative to a distal component axis for the distal component, the flared surface being adjacent the distal component first end or the distal component second end.

7. The device of claim 5,
wherein the proximal component has a flared surface surrounding a portion of the proximal component passage, the flared surface being curved relative to a proximal component axis for the proximal component, the flared surface being adjacent the proximal component first end or the proximal component second end.

8. The device of claim 5,
wherein the proximal component defines a proximal component recess adjacent the proximal component first end, the proximal component recess being coaxial with the proximal component passage;
wherein the proximal component has a flared surface surrounding a portion of the proximal component passage or the proximal component recess, the flared surface being curved relative to a proximal component axis for the proximal component, the flared surface being adjacent the proximal component first end or the proximal component second end.

9. A method for treating a spine, the spine including a first vertebra and a second vertebra, the first vertebra having a first endplate that is adjacent a spinal disc, the second vertebra having a second endplate that is adjacent the spinal disc, the first vertebra having a first pedicle, the method comprising:
(a) providing a proximal component, a distal component, and a guidewire, wherein the proximal component includes first means for anchoring in the first vertebra and the proximal component has a proximal component axis that is substantially straight, wherein the distal component includes second means for anchoring in the second vertebra and the distal component has a distal component axis that is substantially straight;
(b) forming a curved channel that extends through the first vertebra from the first pedicle to the first endplate, the curved channel having a radius of curvature that is less than or equal to 100 percent of a vertebral body height for the first vertebra, wherein the guidewire extends through the curved channel and into the second vertebra;
(c) advancing the distal component over the guidewire through the curved channel to a distal component position for the distal component, wherein the distal component position is entirely within the second vertebra or partially within the second vertebra and partially within the spinal disc;
(d) anchoring the distal component at the second vertebra;
(e) advancing the proximal component over the guidewire through the curved channel to a proximal component position for the proximal component, wherein the proximal component position is entirely within the first vertebra or partially within the first vertebra and partially within the spinal disc; wherein the proximal component axis has an orientation relative to the distal component axis; wherein the orientation changes by at least 40 degrees while the proximal component passes through the curved channel; and
(f) anchoring the proximal component at the first vertebra, wherein the proximal component axis is substantially perpendicular to the first endplate.

10. The method of claim 9,
wherein the proximal component releasably engages the distal component.

11. The method of claim 9, further comprising:
providing an intermediate component; and
prior to step (e), advancing the intermediate component over the guidewire through the curved channel to an intermediate component position for the intermediate component, wherein the intermediate component releasably engages the distal component.

12. A method for treating a spine, the spine including a first vertebra and a second vertebra, the first vertebra having a first endplate that is adjacent a spinal disc, the second vertebra having a second endplate that is adjacent the spinal disc, the first vertebra having a body and a pedicle, the method comprising:
(a) forming a channel that extends through the first vertebra, wherein the channel extends through the pedicle and through the first endplate, the channel having a channel diameter, the channel having a pedicle region, a central region, and an endplate region, wherein the channel diameter for the central region is greater than the channel diameter for the pedicle region and the channel diameter for the central region is greater than the channel diameter for the endplate region;
(b) providing an implant, the implant having an implant diameter, wherein the implant diameter is configured to permit passage of the implant through the pedicle region and through the endplate region;
(c) introducing the implant into the pedicle region; and
(d) advancing the implant through the channel, wherein at least a portion of the implant advances at least to the first endplate.

13. The method of claim 12, further comprising:
installing the implant, wherein the installing comprises positioning the implant at least partially within the spinal disc or at least partially within the first vertebra or at least partially within the second vertebra.

14. The method of claim 12,
wherein the forming comprises:
creating a predecessor channel that extends through the pedicle and through the first endplate, wherein the predecessor channel is coaxial with the channel in at least a portion of the pedicle region and the predecessor channel is coaxial with the channel in at least a portion of the endplate region; and
enlarging the central region for the predecessor channel, wherein the enlarging causes the channel diameter for the central region to be greater than the channel diameter for the pedicle region and the enlarging causes the channel diameter for the central region to be greater than the channel diameter for the endplate region.

15. The method of claim 14,
wherein the enlarging comprises:
cutting or abrading the body where it surrounds the central region of the predecessor channel using a drill, the drill comprising a steerable drill or a flexible drill, the drill comprising a retractable cutting head and a sheath, the retractable cutting head being capable of retracting within the sheath, the sheath dimensioned to be insertable within the predecessor channel, the retractable cutting head capable of emerging from a distal end of the sheath, wherein a cutting head radius for the emerged retractable cutting head is greater than half of the channel diameter for the pedicle region.

16. The method of claim 14,
wherein the enlarging comprises:
advancing a dilator in the predecessor channel to a position within the central region; and
dilating the dilator for displacing cancellous bone of the body that surrounds the central region of the predecessor channel.

17. The method of claim 16,
wherein the dilator comprises a balloon and an inflation line that is connected to the balloon, and wherein the dilating comprises inflating the balloon.

18. The method of claim 16,
wherein the dilator comprises a wedge.

19. The method of claim 12,
wherein the forming comprises:
creating a first predecessor channel and a second predecessor channel, wherein the second predecessor channel diverges from the first predecessor channel in at least a portion of the central region.
